SEQUENCE LISTING (13 sequences)

SEQ ID NO: 1 - DNA, 35187 bp, Artificial Sequence
  Ad20-42-42
catcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgatccacagggtatttaaaccagtcgagcccgtcaagaggccactcttgagtgccagcgagtagagatttctctgagctccgctcccagagtgtgagaaaaatgagacacctgcgcctcctgcctgcaactgtgcccttggacatggccgcattattgctggatgactttgtgagtacagtattggaggatgaactgcaaccaactccgttcgagctgggacccacacttcaggacctctatgatctggaggtagatgcccaggaggacgacccgaacgaagaggctgtgaatttaatatttccagaatctctgattcttcaggctgacatagccagcgaagctgtacctacgccacttcatacaccgactctgtcacccatacctgaattggaagaggaggacgaactagacctccggtgttatgaggaaggttttcctcccagcgattcagaggacgaacagggtgagcagagtatggctataatctcagactatgcttgtgtgcttgtggaagagcattttgtgttggacaatcctgaggtgcccgggcaaggctgtagatcctgccaatatcaccgggatcagaccggagactcaaatgcttcctgcgctctgtgttacatgaaaatgagcttcagctttatttacagtaagtgaagtgaatgtgagaggctgagtgcttaacacatatctgtgtatcgcttgaacagctgtgctaagtgtggtttatttttgttactaggtccggtgtcagaggatgagtcatcaccctcagaagaagaccacccgtctccccctgatctcacagatgacacgcccctgcaagtgttcagacccaccccagtcagagccagtggcgagaggcgagcagctgttgaaaaaattgaggacttgttacatgacatgggtggggatgaacctttggaccttagcttgaaacgccccaggaactaggcgcagctgcgcttagtcatgtgtaaataaagttgtacaataaaagtatatgtgacgcatgcaaggtgtggtttatgactcatgggcggggcttaggcctatataagtggtaacacctgggcactcaggcacagacattcagggagctcctgatggaggtgtggactatccttggagactttaacaagacacgccggcttgtagaggatagttcagacgggtgctccgggttctggagacactggtttggaactcctctatctcgcctggtgtacacagttaagaaggattataacgaggaatttgaaaatctttttgccgactgctctggcctgcttgattctctgaattttggccaccagtcccttttccaggaaagggtcctccacagccttgatttttccagcccagggcgcactacagccggggttgcttttgtggtttttctggttgacaaatggagccagaacacccaactgagcaggggctacattctggacttcgcagccatgcacctgtggagagcctgggtcaggcagcggggacagagaatcttgaactactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagtgaagagggagaggagcgatgggggcaataccgggatgatgaccgagctgacggccagcctgatgaatcgcaagcgcccagagcgcattacctggcacgagctacagatggagtgcagggatgaggtgggcctgatgcaggataaatatggcctggagcagataaaaacccactggttgaacccagatgaggattgggaggaggccattaagaaatatgccaagatagccctgcgcccagattgcaagtatagggtgaccaagaccgtgaatatcagacatgcttgctacatctcggggaacggggcagaggtggtcatcgataccctggacaaggccgccttcaggtgttgcatgatgggaatgagagccggagtgatgaatatgaattccatgatcttcatgaacatgaagttcaatggagagaagtttaatggggtgatgttcatggccaacagccacatgaccctgcacggatgcagtttcttcggcttcaacaatatgtgcgcagaggtctggggcgctgctaagatcaggggatgtaagttttatggatgctggatgggcgtggtcggaagacccaagagcgagatgtctgtgaagcagtgtgtgtttgagaaatgctacctgggagtctctaccgagggcaatgctagagtgagacattgctcttccctggagacgggctgcttctgcctggtgaagggcacagcctctctgaagcataatatggtgaagggctgcacggatgagcgcatgtacaacatgctgacatgcgactcgggggtctgccatatcctgaagaacatccatgtgacctcccacccccggaagaagtggccagtgtttgagaataacctgctgatcaagtgccatatgcacctgggagccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaggtccagggtgcgcgcttgcgagtgcgggggcagacacaccaggatgcaaccggtggccctggatgtgaccgaggagctgaggcccgaccacctggtgatggcatgtaccgggaccgagttcagctccagcggggaggacacagattagaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgcttttgagtaaactctcatccatctggtcagaaaagactatttttttattgtcaagcttggtggcgaaggagccatagagggcgttggagagaagcttggcgatggatctcatggtctgatttttgtcacggtcggcgcgctccttggccgcgatgtttagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtcgcccgcctttgcgcgaacagaaagggggcagcacatcaagcagatgctcgtcgggggggtccgcatcgatggtgaagataccgggacagagttccttgtcaaaatagtctatttttgaggatgcatcatccaaggccatctgccactcacgggcggccattgctcgctcgtaggggttgaggggcggaccccagggcatgggatgcgtgagggcggaggcgtacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtggggcaagcggaccgagtcgcggatgaagtgcgcgtaggagtcttgcagcttggcgacgagctcggcggtgacgaggacgtccatggcgcagtagtccagcgtttcgcggatgatgtcataacccgtctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctctagcgcccactcctggagatgtgggttggcttgcatgaaggaagcccagagctcgcgggccatgagggtctggagctcgtcacgaaagaggcggaactgctggcccacggccatcttttcgggtgtgacgcagtagaaggtgagggggtcccgctcccagcgatcccagcgtaagcgcacggcgagatcgcgagcgagggcgaccagctctgggtcccccgagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgtgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggtcccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcattcgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcgaagacggcgtagttgcgcaggcgctggaagaggtagttgagggtggtggcgatgtgctcggtgacgaagaagtacatgatccagcggcgcagaggcatctcgctgatgtcgccgatggcctccagcctttccatggcctcgtagaaatccacagcgaagttgaaaaactgggcgttgcgggccgagaccgtgagctcgtcttccaggagcctgatgagctcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagtgcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccaacgcccctgagcggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccatgagcgaccagttgacggtctgcaggccgggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctattcctccgtagcctggcggaacgcaaacgggttaggccgcgtgtgtaccccggttcgagtcccctcgaatcaggctggagccgcgactaacgtggtattggcactcccgtctcgacccgagcccgatagctgccaggatacggcggagagcccttttggcggccgaggggggtcgctagacttgaaagcggccgaaaaccctgccgggtagtggctcgcgcccgtagtctggagaagcatcgccagggttgagtcgcggcagaacccggttcgcggacggccgcggagagcgggacttggtcaccccgccgatttaaaaacccacagccagccgacttctccagttacgggagcgagcccccttttttctttttgccagatgcatcccgtcctgcgccaaatgcgtcccaccccccctccggcgaccaccgcaaccgcggccgtagcaggcgccggcgctagccagccacagccacagacagagatggacttggaagagggcgaagggctggcgagattgggggcgccgtctccggagcgacacccccgcgtgcagctgcagaaggacgtgcgcccggcgtacgtgcctgcgcagaacctgttcagggaccgcagcggggaggagcccgaggagatgcgcgactgccgctttcgggcgggcagggagctgcgcgagggtctggaccgccagcgcgtgctgcgcgatgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggtcgctggctgctggagctgatcaacatcttgcagagcatcgtagtgcaggagcgcagcctgagcctggccgagaaagtggcggcgatcaactactcggtgctgagcctgggcaagttttacgcgcgcaagatttacaagacgccatacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaagccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcggccattctggaggcggtggtcccctctcgaaccaaccccacgcacgagaaggtgctggcgatcgtgaacgcgctggcggagaacaaggccatccgtcccgacgaggccggtctggtgtacaacgccctgctggagcgcgtgggccgctacaacagcacaaacgtgcagtccaacctggaccggctggtgacggacgtgcgcgaggccgtggcgcagcgcgagcggttcaagaacgaaggcctgggctcgctggtggcgctgaacgccttcctggcgacgcagccggcgaacgtgccgcgcgggcaggacgattataccaactttataagcgcgctgcggctgatggtgaccgaggtgccccagagcgaggtgtaccagtcgggcccggattactttttccaaactagcagacagggcctgcagacggtgaacctgagccaggcctttaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcacaggtggacgagcagaccttccaggagatcactagcgtgagccgcgcgctggggcagaacgacaccgacagtcttagggccaccctgaacttcttgctgaccaatagacagcagaagatcccggcacagtacgcgctatcggccgaggaggagcgcatcctgagatatgtgcagcagagcgtagggctgttcctgatgcaggagggggccacccccagcgccgcgctggacatgaccgcgcgcaacatggaacctagcatgtacgctgccaaccggccgttcatcaataagctgatggactacctgcaccgcgcggcggccatgaactcggactactttacaaacgccatcctgaacccgcactggctcccgccgccggggttctacacgggcgagtacgatatgcccgaccccaacgacgggttcctgtgggacgacgtggacagcgcggtgttctccccgaccttgcaaaagcgccaggaggcggtgcgcacgcccgcgagcgagggtgcggtgggtcggagcccctttcctagcttagggagtttgcatagcttgccgggctcggtgaacagcggcagggtgagccgaccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccaaactggggcgacggatgttttgaaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttattcggagctggctccgctgtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcacccagagaatgtagctaaagacgatttgagtcaggataagcttgaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcagaaatttcaagctgggctgggacccggtgaccaagttggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaagatctagtagggggcaacatcccagccctcctgaatgtcaaggagtatctgaaggataaggaagaagctggcacagcagatgcaaataccattaaggctcagaatgatgcagtcccaagaggagataactatgcatcagcggcagaagccaaagcagcaggaaaagaaattgagttgaaggccattttgaaagatgattcaaacagaagctacaatgtgatcgagggaaccacagacaccctgtaccgcagttggtacctgtcctatacctacggggaccccgagaagggggtgcagtcgtggacgctgctcaccaccccggacgtcacatgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctatccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttctaccgccgctgccgtgcgtaccaccgtcgacgacgtcatcgactcggtggtcgccgacgcgcgcaactacacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcctgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccaccaacgtgcccatctccatcccctcgcgcaactgggccgccttccgcggctggagtttcacccggctcaagaccaaggaaactccctcccttggctcgggttttgacccctactttgtctactcgggctccatcccctacctcgacgggaccttttacctgaaccataccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttccttgtccagatgctctcccactacaacatcggctaccagggcttccacgtgcccgagggctacaaggaccgcatgtactcattcttccgcaacttccagcccatgagcaggcaggtggtcgatgagatcaactacaaggactacaaagccgtcaccctgcccttccagcacaacaactcgggcttcaccggctatctcgcccccaccatgcgccaggggcagccctaccccgccaacttcccatacccgctcatcggccagacagccgtaccctccgtcacccagaaaaagttcctctgcgacagggtcatgtggcgcatccccttttccagcaacttcatgtccatgggcgccctcaccgacctgggacagaacatgctctatgccaactcggcccacgcgctcgacataacttttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctactacataagcatgagcggctccagcgaacgagagctcgcggccatcgtgcgcgacctgggctgcgggccctactttttgggaacccacgacaagcgcttccctggcttccttgccagcgacaagctggcatgcgccatcgtcaacacggccggccgcgagaccgggggcgtgcactggctcgcctttggctggaatccgcgctcgcgcacctgctacatgtttgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttttgttgcatgtttttgcatgcctttgtgcactggcccgaccgacccatggacggaaaccccaccatgaacttgctgacgggggtgcccaacggcatgctacaatcgccacaggtgctccccaccctcaggcgcaaccaggaggagctctaccgcttcctcgcgcgccactccccttactttcgatcccaccgcgccgccattgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactgaaacttgggctgccacttgaactcggggatcaccagtttgggcactggggtctcggggaaggtctcgctccacatgcgccggctcatctgcagggcgcccagtatgtcgggcgcggagatcttgaaatcgcagttgggaccagtgctctgcgcgcgcgagttgcggtacacggggttgcagcactggaacaccatcaaactggggtgcttcacgctggccagcacgctcttgtcgctgatctgatccttgtccaggtcctcggcgttgctcaggccgaacggggtcatcttgcacagctggcggcccaggaagggcacgctgtgaggcttgtggttacactcgcagtgcacgggcatcagcatcatccccgcgccgcgctgcatattcgggtagagggccttgacaaaggccaagatctgcttgaaagcttgctgggccttggccccctcgctgaagaacaggccgcagctcttcccgctgaactggttattcccacacccggcatcatgcacgcagcagcgcgcgtcatggctggtcagttgcaccacgcttcggccccagcggttctgggtcaccttggccttgctgggctgctccttcaacgcgcgctggccgttctcgctggtcacatccatctccaccacgtggtccttgtggatcatcaccgtcccgtgcagacacttgagctggccctcgacatcggtgcagccgtgttcccacagggcgcagccggtgcactcccaattcttgtgcgcgatcccgctgtggctgaagatgtaaccttgcaacatgcggcccatcacggtgctaaatgatttactggtgctgaaggtcagttgcaggccgcgggcctcctcgttcatccatgtctgacacattttctggaagatctcggtctgctcgggcatgagcttgtaggcatcgcgcaggccgctgtcgacgcggtagcgttccatcagcacgttcatggtatccatgcccttctcccaggacgagaccagaggcagactcagggggttgcgcacgttcaggataccgggggtcgcgggctcgacgatgcgttttccgtcctttccttccttcaacagaaccggcggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcggtccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctgaaacacctgcagcgccagtccctcatcctccgggacgccctagccgaccggagcgaaacccccctcagcgtcgaggagctgtgtcgggcctacgagctcaacctcttctcgccgcgcatgccccccaaacgccagcccaatggcacatgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctccttactctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagcttatcatgccccacatcgaggccctcgatgaaagtcaggagcagcgccccgaggacgcccggcccgtggtcagcgacgagatgctcgcgcgctggctcgggacccgcgacccccaggctttggaacagcggcgcaagctcatgctggccgtggtcctggtcaccctcgagctggaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctgggccagaccgtgctccactctaccctgaagggcgaggcgcggcgggactacgtccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgagacagacgctgaaaggcgggctgcccgacttcatgagccagagcatgttgcaaaactaccgcactttcattctcgagcgatctgggatgctgcccgccacctgcaacgccttcccctccgactttgtaccgctgagctaccgcgagtgtcccccgccgctgtggagccactgctacctcttgcagctggccaactacatcgcctaccactcggacgtgatcgaggacgtgagcggcgaggggctgctcgagtgccactgtcgctgcaacctgtgctccccgcatcgctccctggtctgcaacccccagctcctgagcgagacccaggtcatcggtaccttcgagctgcaaggtccgcaggagtccaccgctccgctgaaactcacgccggggttgtggacttccgcgtacctgcgcaaatttgtacccgaagactaccacgcccatgagataaagttctttgaggaccaatcgcgcccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagagcagcccgtcgccgcaccatccgcgccggcagccccgccggtcacggatacaacctccgctccggtcaagcctcctcgtagatgggatcaaatgaagggtgacggtaagcacgagcggcagggctaccgatcatggagggcccacaaagccgcgatcatcgcctgcttgcaagactgcggggggaacatcgcttttgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacaggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagcttaacgacgagttggtgagctcttcaatcggtctgcgaccggacggagtgttccaactagccggagccgggagatcctccttcactccccaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgatgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgttgtactctgtgtgctgagtttaataaaagctaaactcctacaatactctgggatcccgtgtcgtcgcactcgcaacgagatcttcaacctcaccaaccagactgaggtaaaactcaactgcagaccggggggcaaatacatcctctggctctttgaaaacacttccttcgcagtctccaacgcctgcgccaacgacggtattgaaatacccaacaaccttaccagtggactaacttacactaccagaaagactaagctagtactctacaatccttttgtagagggaacctaccactgccagagcggaccttgcttccacactttcactttggtgaacgttaccgacagcagcacagtcgctccagaaacatctaacctttttgatactaacactcctaaaaccggaggtgagctctgggttccctctttaacagaggggggtaaacatattgaagcggttgggtatttgattttaggggtggtcctgggtgggtgcatagcggtgctgtattaccttccttgctggatcgaaatcaaaatctttatctgctgggtcatacattgttgggaggaaccatgaaggggctcttgctgattatcctttccctggttgggggtgtactgtcatgccacgaacagccacgatgtaacatcaccacaggcaatgagaggagtgtgatatgcacagtagtcatcaaatgcgagcatgcatgtcctctcaacatcacattcaagaataagaccatgggaaattcatgggtgggcgattgggaaccaggagatgagcagaactacacggtcactgtccatggtagcgatgggaatcacactttcggtttcaaattcatttttgaagtcatgtgtgatattacactgcatgtggctagacttcatggcttgtggccccctaccaaggagaacatggttgggttttctttggcttttgtgatcatggcctgctttatgtcaggtctgctggtaggggctttagtatggttcctgaagcacaagcctaggtatggaaatgaggagaaggaaaaattgctataaatctttttctcttcgcagaaccatgaatactttgaccagtgtcgtgctgctctctcttcttgtagcttttagtcaggcaggatttcatactatcaatgctacatggtgggctaatgtaactttagtgggaccctcagatacgccagtcacatggtatgataaacagggaatgcagttctgtgatggaaatacagttaaaaatcctcaaataagacatgagtgtaatgagcaaaaccttacactaattcatgtgaacaaaacccatgaaaggacatacatgggttataatagacagagtactcataaggaagactataaagtcatagttataccgcctcctcctgctactgtaaagccacagtcaggtccagagtatgtatatgttaatatgggagagaacaaaaccttagttggacctccaggaataccagttacttggtatgacggagaaggaaataaattctgcgatggagaaaaagttgaacatgcagaatttaatcatacatgtgacgagcaaaatcttacactgttgtttataaatcttacacatgatggggcttatcttggctataatcaccagggaactaaaagaacttggtatgaggttgtagtgacagatggttttccaaaatcaggggagatgaaaatcgaagatcagagtagacaaacagaacaaaaacaaaatgagcataaacagggtgggcagaaacaggaggggcaaaaagagacaagtcaaaagaaagataatgacaaacagaaggcgacacacaggaggccatcaaaactaaagccgcacacacctgaagcaaaactgattacagtttctagtgggtctaacttaacattacttgggccagatggaaaggtcacttggtatgatgatgatttaaaaagaccatgcgagcctgggtataagttagggtgtaagtgtgacaatcaaaacctaacgctaatcaatgtaactaaactttatgagggagtttactatggtactaatgacagaggcaacagcaaaagatacagagtaaaagtaaacactactaattctcaaagtgtgaaaattcagccatacaacaggtctactactcctgatcagaaacacagatttgaattgcaaattgattctaatcaagacaatgacaaaattccatcaaccactgtggcaatcgtggtgggagtgattgcgggcttcataactataatcattgtcattatgtgctacatctgctgccgcaagcgtcccagggcatacaatcatatggtagacccactacttagcttctcttactgagactcagtcactttcatttcagaaccatgaaggctttcacagcttgcgttctgattagcatagtcacattagtatcagctgattacaaacaagttcaagttagcagaggaggaaatattacattagatggaccattcgataatactacatggacaagatatcataatgatggacataaaaatggttggatgaaaatttgcacatggactggagcaacatataaatgtcacaataatggaagcattactatttttgctttcaacattacatccggagtttacaaagcagaagggtataaaaaagaggttagaacattttcatctagaaatcaaaaacatacaattgaagattctggagattatgaacaacaaaaaatatatctatataatctaacaataattgaaccgccaactactaaagcacccaccacagttagaacaaccacgcagacaactactagggaaacaacacatccaaccaccacagtcagtacaactcacactacacatctagacactacagtgcagaatactactttattgattgggtttttactaagaggaaatgaaagtactactgatcagacagaggctacctcaagtgccttcagcagcactgcaaatttaacttcgcttgcttcggtaaatgaaacgatcgtgccgatgatgtatggccaaccttactcaggtttggatattcaaattacttttctggttgtctgtgggatctttattcttgtggttcttttgtactttgtctgctgcaaagccagagaaaaatctaggaggcccatctacaggccagtaatcggggatcctcagcctctccaagtggaagggggtctaaggaatcttctcttctctttttcagtatggtgattcagccatgattcctaggttcttcctatttaacatcctcttctgtctattcaacgtgtgcgctgccttcgcggccgtctcgcacgcctcgcccgactgtcttgggcccttccccacctaccttctttttgccctgctcacctgcacctgcgtctgcagcattgtctgcctggtcgtcaccttcctgcagctcatcgactggtgctgcgcgcgctacaattatctccaccacagtcccgaatacagggacgagaacgtagccagaatcttaaggctcatttgaccatgcagactctgctcatactgctatccctcctctcccctgccctcgctgatgatgattactctaagtgcaaatttgtggagctatggaatttcttagactgctatgatgctaaaatggatatgccatcctattacttggtgattgtggggatagtcatggtctgctcctgcactttctttgccatcatgatctacccctgttttgatctcggctggaactctgttgaggcattcacatacacactagaaagcagttcactagcttccacgccgccacccacaccgcctccccgcagaaatcagtttcccatgattcagtacttagaagagccccctccccggcccccttccactgttagctactttcacataaccggcggcgatgactgacaaccacctggacctcgagatggacggccaggcctccgagcagcgcatcctgcaactgcgcgtccgtcagcagcaggagcgggccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctcctcaagaccctttgcggacttcgcgacctcctccccatgaactgattgattaaagcccaaaaaaccaatcaaaccccttcccattagccccaaataaacaatcattggaaataatcattcaataaagatcacttacttgaaatctgaaagtatgtctctggtgtagttgtttagcagcacctcggtaccctcctcccagctctggtactccagtccccggcgggcggcgaactttctccacaccttgaaagggatgtcaaattcctggtccacaattttcattgttttccctctcagatgtcaaagaggcttcgggtggaagatgacttcaaccccgtctacccctatgactacgcgcggaatcagaatatccccttcctcactcccccctttgtctcctccgatggattccaaaacttcccccctggtgtcctgtcactcaaattggctgacccaatcgctatcaccaatggtgatgtctcactcaaggtgggagggggacttactttacaagatggaaccggaaaactaactatcgacaccaagactcccttgcaagttgcaaataataaattagaacttgcgtttgatgcaccattgtatgaaaaaaatggaaaacttgctttaaaaacaggccatggattagctgttttaaccaaagacattggcataccagaattaattggatcccttgtgatcttaactggaaaaggaattgggacaggtactgttgcaggaggaggaactatagatgtaagactgggtgatgatggaggtttatcatttgataaaaagggtgatctagtagcctggaataaaaaaaatgacaggcgcactttgtggacaacacctgatccatccccaaactgcagagtatcagaagataaagattcaaaactaactttaattcttacaaaatgtggaagtcagatcctagcaagtttttcactgcttgtagtcaaagggacgtatgcaactgttgataaaaatacaactaataaacaatttagcattaaactactgtttgatgcaaatggaaagcttaaaagcgaatcaaaccttagtggttattggaactatagaagtgataatagtgttgttagtactccctatgacaatgcagtgcctttcatgccaaataccacggcttatccaaaaatcattaatagtacaactgatcctgaaaataagaaaagttcggctaaaaaaactattgttggcaatgtctacctagaaggtaatgcgggtcaaccagtagccgttgctattagtttcaataaggaaactactgctgactattcaataacatttgactttgcgtggagcaaagcttatgaaacccctgtgccttttgacacctcctccatgacattctcatatattgcccaggaaaatcaagacaaaggcgaataaagtgttttgaaatgaacttatgtatctttattgatttttacaccagcacgggtagtcagtctcccaccaccagcccatttcacagtgtacacggttctctcagcacgggtggccttaaatagggaaatgttctgattagtgcgggaactggacttggggtctataatccacacagtttcctggcgagccaaacgggggtcggtgattgagatgaagccgtcctctgaaaagtcatccaagcgggcctcacagtccaaggtcacagtctggtgaaacgagaagaacgcacagattcatactcggaaaacaggatgggtctgtgcctctccatcagcgccctcaacagtctctgccgccggggctcggtgcggctgctgcagatgggatcgggatcgcaagtctctctgactatgatccccacagccttcagcatcagtctcctggtgcgtcgggcacagcaccgcatcctgatctctgccatgttctcacagtaagtgcagcacataatcaccatgttattcagcagcccataattcagggtgctccagccaaaactcatgttggggatgatggaacccacgtgaccatcgtaccagatgcggcagtatatcagatgcctgcccctcatgaacacactgcccatatacatgatctctttgggcatgtctctgttcacaatctgacggtaccagggaaagcgctggttgaacatgcacccgtaaatgactctcctgaaccacacggccagcagggtgcctcccgcccgacactgcagggagcccggggatgaacagtggcaatgcaggatccagcgctcgtacccgctcaccatctgagctctcaccaagtccagggtagcggggcacaggcacactgacatacatctttttaaaatttttatttcctctggagtcaagatcatatcccaggggactggaaactcttggagcagggtaaagccagcagcacatggtaatccacggacagaacttacattatgataatctgcatgatcacaatcaggcaacaggggatgttgttcagtcagtgaagccctggtttcctcatcagatcgtggtaaacgggccctgcgatatggatgatggcggagcgagctggattgaatctcggtttgcattgtagtggattctcttgcgtaccttgtcgtacttctgccagcagaaatgggcccttgaacagcagatacccctcctgcggccgtcctttcgctgctgccgctcagtcatccaactgaagtacatccattctcgaagattctggagaagttcctctgcatctgatgaaataaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccgaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgtttttcgcgaaaacacttccgcgttctcacttcctcgtatcgatttcgtgacttaacttccgggttcccacgttacgtcacttctgcccttacatgtaactgagtcgtagggcgccatcttgcccacgtccaaaatggcttccatgtccagccacgcctccgcggcgaccgttagccgtgcgtcgtgacgtcatttgcatcgtcttctctcgtccaatcagcgtcggctccgccctaaattcaaaagctcatttgcatattaacttttgtttactttgtggggtatattattgatgatg

SEQ ID NO: 2 - DNA, 6150 bp, Artificial Sequence
  pAdAptAd20-42-42.Empty
ttaattaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagattaattaattcgaacccataatacccataatagctgtttgccatcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgatggcaaacagctattatgggtattatgggttcgaa

SEQ ID NO: 3 - DNA, 9350 bp, Artificial Sequence
  pAdAptAd20-42-42.LacZ
ttaattaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtacctctaggcttttgcaaaaagctttgcaaagatggataaagttttccggaattccgccaaaaagaagagaaaggtagaagaccccaaggactttccttcagaattgctaagttttttgagtccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggattctgaacttgtttatggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagattaattaattcgaacccataatacccataatagctgtttgccatcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgatggcaaacagctattatgggtattatgggttcgaa

SEQ ID NO: 4 - DNA, 7800 bp, Artificial Sequence
  pAdAptAd20-42-42.Luc+
ttaattaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagattaattaattcgaacccataatacccataatagctgtttgccatcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgatggcaaacagctattatgggtattatgggttcgaa

SEQ ID NO: 5 - DNA, 6901 bp, Artificial Sequence
  pAdAptAd20-42-42.eGFP
ttaattaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcgctagcgttaacggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagattaattaattcgaacccataatacccataatagctgtttgccatcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgatggcaaacagctattatgggtattatgggttcgaa

SEQ ID NO: 6 - DNA, 18499 bp, Artificial Sequence
  pBR.Ad20-42-42.SbfI final intermediate
attaaaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgcttttgagtaaactctcatccatctggtcagaaaagactatttttttattgtcaagcttggtggcgaaggagccatagagggcgttggagagaagcttggcgatggatctcatggtctgatttttgtcacggtcggcgcgctccttggccgcgatgtttagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtcgcccgcctttgcgcgaacagaaagggggcagcacatcaagcagatgctcgtcgggggggtccgcatcgatggtgaagataccgggacagagttccttgtcaaaatagtctatttttgaggatgcatcatccaaggccatctgccactcacgggcggccattgctcgctcgtaggggttgaggggcggaccccagggcatgggatgcgtgagggcggaggcgtacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtggggcaagcggaccgagtcgcggatgaagtgcgcgtaggagtcttgcagcttggcgacgagctcggcggtgacgaggacgtccatggcgcagtagtccagcgtttcgcggatgatgtcataacccgtctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctctagcgcccactcctggagatgtgggttggcttgcatgaaggaagcccagagctcgcgggccatgagggtctggagctcgtcacgaaagaggcggaactgctggcccacggccatcttttcgggtgtgacgcagtagaaggtgagggggtcccgctcccagcgatcccagcgtaagcgcacggcgagatcgcgagcgagggcgaccagctctgggtcccccgagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgtgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggtcccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcattcgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcgaagacggcgtagttgcgcaggcgctggaagaggtagttgagggtggtggcgatgtgctcggtgacgaagaagtacatgatccagcggcgcagaggcatctcgctgatgtcgccgatggcctccagcctttccatggcctcgtagaaatccacagcgaagttgaaaaactgggcgttgcgggccgagaccgtgagctcgtcttccaggagcctgatgagctcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagtgcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccaacgcccctgagcggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccatgagcgaccagttgacggtctgcaggccgggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctattcctccgtagcctggcggaacgcaaacgggttaggccgcgtgtgtaccccggttcgagtcccctcgaatcaggctggagccgcgactaacgtggtattggcactcccgtctcgacccgagcccgatagctgccaggatacggcggagagcccttttggcggccgaggggggtcgctagacttgaaagcggccgaaaaccctgccgggtagtggctcgcgcccgtagtctggagaagcatcgccagggttgagtcgcggcagaacccggttcgcggacggccgcggagagcgggacttggtcaccccgccgatttaaaaacccacagccagccgacttctccagttacgggagcgagcccccttttttctttttgccagatgcatcccgtcctgcgccaaatgcgtcccaccccccctccggcgaccaccgcaaccgcggccgtagcaggcgccggcgctagccagccacagccacagacagagatggacttggaagagggcgaagggctggcgagattgggggcgccgtctccggagcgacacccccgcgtgcagctgcagaaggacgtgcgcccggcgtacgtgcctgcgcagaacctgttcagggaccgcagcggggaggagcccgaggagatgcgcgactgccgctttcgggcgggcagggagctgcgcgagggtctggaccgccagcgcgtgctgcgcgatgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggtcgctggctgctggagctgatcaacatcttgcagagcatcgtagtgcaggagcgcagcctgagcctggccgagaaagtggcggcgatcaactactcggtgctgagcctgggcaagttttacgcgcgcaagatttacaagacgccatacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaagccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcggccattctggaggcggtggtcccctctcgaaccaaccccacgcacgagaaggtgctggcgatcgtgaacgcgctggcggagaacaaggccatccgtcccgacgaggccggtctggtgtacaacgccctgctggagcgcgtgggccgctacaacagcacaaacgtgcagtccaacctggaccggctggtgacggacgtgcgcgaggccgtggcgcagcgcgagcggttcaagaacgaaggcctgggctcgctggtggcgctgaacgccttcctggcgacgcagccggcgaacgtgccgcgcgggcaggacgattataccaactttataagcgcgctgcggctgatggtgaccgaggtgccccagagcgaggtgtaccagtcgggcccggattactttttccaaactagcagacagggcctgcagacggtgaacctgagccaggcctttaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcacaggtggacgagcagaccttccaggagatcactagcgtgagccgcgcgctggggcagaacgacaccgacagtcttagggccaccctgaacttcttgctgaccaatagacagcagaagatcccggcacagtacgcgctatcggccgaggaggagcgcatcctgagatatgtgcagcagagcgtagggctgttcctgatgcaggagggggccacccccagcgccgcgctggacatgaccgcgcgcaacatggaacctagcatgtacgctgccaaccggccgttcatcaataagctgatggactacctgcaccgcgcggcggccatgaactcggactactttacaaacgccatcctgaacccgcactggctcccgccgccggggttctacacgggcgagtacgatatgcccgaccccaacgacgggttcctgtgggacgacgtggacagcgcggtgttctccccgaccttgcaaaagcgccaggaggcggtgcgcacgcccgcgagcgagggtgcggtgggtcggagcccctttcctagcttagggagtttgcatagcttgccgggctcggtgaacagcggcagggtgagccgaccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccaaactggggcgacggatgttttgaaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttattcggagctggctccgctgtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcacccagagaatgtagctaaagacgatttgagtcaggataagcttgaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcagaaatttcaagctgggctgggacccggtgaccaagttggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaagatctagtagggggcaacatcccagccctcctgaatgtcaaggagtatctgaaggataaggaagaagctggcacagcagatgcaaataccattaaggctcagaatgatgcagtcccaagaggagataactatgcatcagcggcagaagccaaagcagcaggaaaagaaattgagttgaaggccattttgaaagatgattcaaacagaagctacaatgtgatcgagggaaccacagacaccctgtaccgcagttggtacctgtcctatacctacggggaccccgagaagggggtgcagtcgtggacgctgctcaccaccccggacgtcacatgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctatccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttctaccgccgctgccgtgcgtaccaccgtcgacgacgtcatcgactcggtggtcgccgacgcgcgcaactacacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcctgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccattaattaattcgaacccataatacccataatagctgtttgccatcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatggcaaacagctattatgggtattatgggttcgaatta

SEQ ID NO: 7 - DNA, 17834 bp, Artificial Sequence
  pBrAd20-42-42 SrfI-rITR_dE3_5orf6
tgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccaccaacgtgcccatctccatcccctcgcgcaactgggccgccttccgcggctggagtttcacccggctcaagaccaaggaaactccctcccttggctcgggttttgacccctactttgtctactcgggctccatcccctacctcgacgggaccttttacctgaaccataccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttccttgtccagatgctctcccactacaacatcggctaccagggcttccacgtgcccgagggctacaaggaccgcatgtactcattcttccgcaacttccagcccatgagcaggcaggtggtcgatgagatcaactacaaggactacaaagccgtcaccctgcccttccagcacaacaactcgggcttcaccggctatctcgcccccaccatgcgccaggggcagccctaccccgccaacttcccatacccgctcatcggccagacagccgtaccctccgtcacccagaaaaagttcctctgcgacagggtcatgtggcgcatccccttttccagcaacttcatgtccatgggcgccctcaccgacctgggacagaacatgctctatgccaactcggcccacgcgctcgacataacttttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctactacataagcatgagcggctccagcgaacgagagctcgcggccatcgtgcgcgacctgggctgcgggccctactttttgggaacccacgacaagcgcttccctggcttccttgccagcgacaagctggcatgcgccatcgtcaacacggccggccgcgagaccgggggcgtgcactggctcgcctttggctggaatccgcgctcgcgcacctgctacatgtttgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttttgttgcatgtttttgcatgcctttgtgcactggcccgaccgacccatggacggaaaccccaccatgaacttgctgacgggggtgcccaacggcatgctacaatcgccacaggtgctccccaccctcaggcgcaaccaggaggagctctaccgcttcctcgcgcgccactccccttactttcgatcccaccgcgccgccattgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactgaaacttgggctgccacttgaactcggggatcaccagtttgggcactggggtctcggggaaggtctcgctccacatgcgccggctcatctgcagggcgcccagtatgtcgggcgcggagatcttgaaatcgcagttgggaccagtgctctgcgcgcgcgagttgcggtacacggggttgcagcactggaacaccatcaaactggggtgcttcacgctggccagcacgctcttgtcgctgatctgatccttgtccaggtcctcggcgttgctcaggccgaacggggtcatcttgcacagctggcggcccaggaagggcacgctgtgaggcttgtggttacactcgcagtgcacgggcatcagcatcatccccgcgccgcgctgcatattcgggtagagggccttgacaaaggccaagatctgcttgaaagcttgctgggccttggccccctcgctgaagaacaggccgcagctcttcccgctgaactggttattcccacacccggcatcatgcacgcagcagcgcgcgtcatggctggtcagttgcaccacgcttcggccccagcggttctgggtcaccttggccttgctgggctgctccttcaacgcgcgctggccgttctcgctggtcacatccatctccaccacgtggtccttgtggatcatcaccgtcccgtgcagacacttgagctggccctcgacatcggtgcagccgtgttcccacagggcgcagccggtgcactcccaattcttgtgcgcgatcccgctgtggctgaagatgtaaccttgcaacatgcggcccatcacggtgctaaatgatttactggtgctgaaggtcagttgcaggccgcgggcctcctcgttcatccatgtctgacacattttctggaagatctcggtctgctcgggcatgagcttgtaggcatcgcgcaggccgctgtcgacgcggtagcgttccatcagcacgttcatggtatccatgcccttctcccaggacgagaccagaggcagactcagggggttgcgcacgttcaggataccgggggtcgcgggctcgacgatgcgttttccgtcctttccttccttcaacagaaccggcggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcggtccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctgaaacacctgcagcgccagtccctcatcctccgggacgccctagccgaccggagcgaaacccccctcagcgtcgaggagctgtgtcgggcctacgagctcaacctcttctcgccgcgcatgccccccaaacgccagcccaatggcacatgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctccttactctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagcttatcatgccccacatcgaggccctcgatgaaagtcaggagcagcgccccgaggacgcccggcccgtggtcagcgacgagatgctcgcgcgctggctcgggacccgcgacccccaggctttggaacagcggcgcaagctcatgctggccgtggtcctggtcaccctcgagctggaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctgggccagaccgtgctccactctaccctgaagggcgaggcgcggcgggactacgtccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgagacagacgctgaaaggcgggctgcccgacttcatgagccagagcatgttgcaaaactaccgcactttcattctcgagcgatctgggatgctgcccgccacctgcaacgccttcccctccgactttgtaccgctgagctaccgcgagtgtcccccgccgctgtggagccactgctacctcttgcagctggccaactacatcgcctaccactcggacgtgatcgaggacgtgagcggcgaggggctgctcgagtgccactgtcgctgcaacctgtgctccccgcatcgctccctggtctgcaacccccagctcctgagcgagacccaggtcatcggtaccttcgagctgcaaggtccgcaggagtccaccgctccgctgaaactcacgccggggttgtggacttccgcgtacctgcgcaaatttgtacccgaagactaccacgcccatgagataaagttctttgaggaccaatcgcgcccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagagcagcccgtcgccgcaccatccgcgccggcagccccgccggtcacggatacaacctccgctccggtcaagcctcctcgtagatgggatcaaatgaagggtgacggtaagcacgagcggcagggctaccgatcatggagggcccacaaagccgcgatcatcgcctgcttgcaagactgcggggggaacatcgcttttgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacaggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagcttaacgacgagttggtgagctcttcaatcggtctgcgaccggacggagtgttccaactagccggagccgggagatcctccttcactccccaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgatgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgttgtactctgtgtgctgagtttaataaaagctaaactcctacaatactctgggatcccgtgtcgtcgcactcgcaacgagatcttcaacctcaccaaccagactgaggtaaaactcaactgcagaccggggggcaaatacatcctctggctctttgaaaacacttccttcgcagtctccaacgcctgctgcactagtccatgaactgattgattaaagcccaaaaaaccaatcaaaccccttcccattagccccaaataaacaatcattggaaataatcattcaataaagatcacttacttgaaatctgaaagtatgtctctggtgtagttgtttagcagcacctcggtaccctcctcccagctctggtactccagtccccggcgggcggcgaactttctccacaccttgaaagggatgtcaaattcctggtccacaattttcattgttttccctctcagatgtcaaagaggcttcgggtggaagatgacttcaaccccgtctacccctatgactacgcgcggaatcagaatatccccttcctcactcccccctttgtctcctccgatggattccaaaacttcccccctggtgtcctgtcactcaaattggctgacccaatcgctatcaccaatggtgatgtctcactcaaggtgggagggggacttactttacaagatggaaccggaaaactaactatcgacaccaagactcccttgcaagttgcaaataataaattagaacttgcgtttgatgcaccattgtatgaaaaaaatggaaaacttgctttaaaaacaggccatggattagctgttttaaccaaagacattggcataccagaattaattggatcccttgtgatcttaactggaaaaggaattgggacaggtactgttgcaggaggaggaactatagatgtaagactgggtgatgatggaggtttatcatttgataaaaagggtgatctagtagcctggaataaaaaaaatgacaggcgcactttgtggacaacacctgatccatccccaaactgcagagtatcagaagataaagattcaaaactaactttaattcttacaaaatgtggaagtcagatcctagcaagtttttcactgcttgtagtcaaagggacgtatgcaactgttgataaaaatacaactaataaacaatttagcattaaactactgtttgatgcaaatggaaagcttaaaagcgaatcaaaccttagtggttattggaactatagaagtgataatagtgttgttagtactccctatgacaatgcagtgcctttcatgccaaataccacggcttatccaaaaatcattaatagtacaactgatcctgaaaataagaaaagttcggctaaaaaaactattgttggcaatgtctacctagaaggtaatgcgggtcaaccagtagccgttgctattagtttcaataaggaaactactgctgactattcaataacatttgactttgcgtggagcaaagcttatgaaacccctgtgccttttgacacctcctccatgacattctcatatattgcccaggaaaatcaagacaaaggcgaataaagtgttttgaaatgaacttatgtatctttattgatttttacaccagcacgggtagtcagtctcccaccaccagcccatttcacagtgtaaacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatttcgctgctgccgctcagtcatccaactgaagtacatccattctcgaagattctggagaagttcctctgcatctgatgaaataaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccgaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgtttttcgcgaaaacacttccgcgttctcacttcctcgtatcgatttcgtgacttaacttccgggttcccacgttacgtcacttctgcccttacatgtaactgagtcgtagggcgccatcttgcccacgtccaaaatggcttccatgtccagccacgcctccgcggcgaccgttagccgtgcgtcgtgacgtcatttgcatcgtcttctctcgtccaatcagcgtcggctccgccctaaattcaaaagctcatttgcatattaacttttgtttactttgtggggtatattattgatgatgttaattaattcgaacccataatacccataatagctgtttgccatcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgatggcaaacagctattatgggtattatgggttcgaattaattaaactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcc

SEQ ID NO: 8 - DNA, 32430 bp, Artificial Sequence
  Ad20-42-42.LacZ.5ORF6
attaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtacctctaggcttttgcaaaaagctttgcaaagatggataaagttttccggaattccgccaaaaagaagagaaaggtagaagaccccaaggactttccttcagaattgctaagttttttgagtccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggattctgaacttgtttatggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgcttttgagtaaactctcatccatctggtcagaaaagactatttttttattgtcaagcttggtggcgaaggagccatagagggcgttggagagaagcttggcgatggatctcatggtctgatttttgtcacggtcggcgcgctccttggccgcgatgtttagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtcgcccgcctttgcgcgaacagaaagggggcagcacatcaagcagatgctcgtcgggggggtccgcatcgatggtgaagataccgggacagagttccttgtcaaaatagtctatttttgaggatgcatcatccaaggccatctgccactcacgggcggccattgctcgctcgtaggggttgaggggcggaccccagggcatgggatgcgtgagggcggaggcgtacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtggggcaagcggaccgagtcgcggatgaagtgcgcgtaggagtcttgcagcttggcgacgagctcggcggtgacgaggacgtccatggcgcagtagtccagcgtttcgcggatgatgtcataacccgtctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctctagcgcccactcctggagatgtgggttggcttgcatgaaggaagcccagagctcgcgggccatgagggtctggagctcgtcacgaaagaggcggaactgctggcccacggccatcttttcgggtgtgacgcagtagaaggtgagggggtcccgctcccagcgatcccagcgtaagcgcacggcgagatcgcgagcgagggcgaccagctctgggtcccccgagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgtgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggtcccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcattcgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcgaagacggcgtagttgcgcaggcgctggaagaggtagttgagggtggtggcgatgtgctcggtgacgaagaagtacatgatccagcggcgcagaggcatctcgctgatgtcgccgatggcctccagcctttccatggcctcgtagaaatccacagcgaagttgaaaaactgggcgttgcgggccgagaccgtgagctcgtcttccaggagcctgatgagctcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagtgcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccaacgcccctgagcggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccatgagcgaccagttgacggtctgcaggccgggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctattcctccgtagcctggcggaacgcaaacgggttaggccgcgtgtgtaccccggttcgagtcccctcgaatcaggctggagccgcgactaacgtggtattggcactcccgtctcgacccgagcccgatagctgccaggatacggcggagagcccttttggcggccgaggggggtcgctagacttgaaagcggccgaaaaccctgccgggtagtggctcgcgcccgtagtctggagaagcatcgccagggttgagtcgcggcagaacccggttcgcggacggccgcggagagcgggacttggtcaccccgccgatttaaaaacccacagccagccgacttctccagttacgggagcgagcccccttttttctttttgccagatgcatcccgtcctgcgccaaatgcgtcccaccccccctccggcgaccaccgcaaccgcggccgtagcaggcgccggcgctagccagccacagccacagacagagatggacttggaagagggcgaagggctggcgagattgggggcgccgtctccggagcgacacccccgcgtgcagctgcagaaggacgtgcgcccggcgtacgtgcctgcgcagaacctgttcagggaccgcagcggggaggagcccgaggagatgcgcgactgccgctttcgggcgggcagggagctgcgcgagggtctggaccgccagcgcgtgctgcgcgatgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggtcgctggctgctggagctgatcaacatcttgcagagcatcgtagtgcaggagcgcagcctgagcctggccgagaaagtggcggcgatcaactactcggtgctgagcctgggcaagttttacgcgcgcaagatttacaagacgccatacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaagccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcggccattctggaggcggtggtcccctctcgaaccaaccccacgcacgagaaggtgctggcgatcgtgaacgcgctggcggagaacaaggccatccgtcccgacgaggccggtctggtgtacaacgccctgctggagcgcgtgggccgctacaacagcacaaacgtgcagtccaacctggaccggctggtgacggacgtgcgcgaggccgtggcgcagcgcgagcggttcaagaacgaaggcctgggctcgctggtggcgctgaacgccttcctggcgacgcagccggcgaacgtgccgcgcgggcaggacgattataccaactttataagcgcgctgcggctgatggtgaccgaggtgccccagagcgaggtgtaccagtcgggcccggattactttttccaaactagcagacagggcctgcagacggtgaacctgagccaggcctttaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcacaggtggacgagcagaccttccaggagatcactagcgtgagccgcgcgctggggcagaacgacaccgacagtcttagggccaccctgaacttcttgctgaccaatagacagcagaagatcccggcacagtacgcgctatcggccgaggaggagcgcatcctgagatatgtgcagcagagcgtagggctgttcctgatgcaggagggggccacccccagcgccgcgctggacatgaccgcgcgcaacatggaacctagcatgtacgctgccaaccggccgttcatcaataagctgatggactacctgcaccgcgcggcggccatgaactcggactactttacaaacgccatcctgaacccgcactggctcccgccgccggggttctacacgggcgagtacgatatgcccgaccccaacgacgggttcctgtgggacgacgtggacagcgcggtgttctccccgaccttgcaaaagcgccaggaggcggtgcgcacgcccgcgagcgagggtgcggtgggtcggagcccctttcctagcttagggagtttgcatagcttgccgggctcggtgaacagcggcagggtgagccgaccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccaaactggggcgacggatgttttgaaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttattcggagctggctccgctgtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcacccagagaatgtagctaaagacgatttgagtcaggataagcttgaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcagaaatttcaagctgggctgggacccggtgaccaagttggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaagatctagtagggggcaacatcccagccctcctgaatgtcaaggagtatctgaaggataaggaagaagctggcacagcagatgcaaataccattaaggctcagaatgatgcagtcccaagaggagataactatgcatcagcggcagaagccaaagcagcaggaaaagaaattgagttgaaggccattttgaaagatgattcaaacagaagctacaatgtgatcgagggaaccacagacaccctgtaccgcagttggtacctgtcctatacctacggggaccccgagaagggggtgcagtcgtggacgctgctcaccaccccggacgtcacatgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctatccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttctaccgccgctgccgtgcgtaccaccgtcgacgacgtcatcgactcggtggtcgccgacgcgcgcaactacacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcctgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccaccaacgtgcccatctccatcccctcgcgcaactgggccgccttccgcggctggagtttcacccggctcaagaccaaggaaactccctcccttggctcgggttttgacccctactttgtctactcgggctccatcccctacctcgacgggaccttttacctgaaccataccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttccttgtccagatgctctcccactacaacatcggctaccagggcttccacgtgcccgagggctacaaggaccgcatgtactcattcttccgcaacttccagcccatgagcaggcaggtggtcgatgagatcaactacaaggactacaaagccgtcaccctgcccttccagcacaacaactcgggcttcaccggctatctcgcccccaccatgcgccaggggcagccctaccccgccaacttcccatacccgctcatcggccagacagccgtaccctccgtcacccagaaaaagttcctctgcgacagggtcatgtggcgcatccccttttccagcaacttcatgtccatgggcgccctcaccgacctgggacagaacatgctctatgccaactcggcccacgcgctcgacataacttttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctactacataagcatgagcggctccagcgaacgagagctcgcggccatcgtgcgcgacctgggctgcgggccctactttttgggaacccacgacaagcgcttccctggcttccttgccagcgacaagctggcatgcgccatcgtcaacacggccggccgcgagaccgggggcgtgcactggctcgcctttggctggaatccgcgctcgcgcacctgctacatgtttgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttttgttgcatgtttttgcatgcctttgtgcactggcccgaccgacccatggacggaaaccccaccatgaacttgctgacgggggtgcccaacggcatgctacaatcgccacaggtgctccccaccctcaggcgcaaccaggaggagctctaccgcttcctcgcgcgccactccccttactttcgatcccaccgcgccgccattgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactgaaacttgggctgccacttgaactcggggatcaccagtttgggcactggggtctcggggaaggtctcgctccacatgcgccggctcatctgcagggcgcccagtatgtcgggcgcggagatcttgaaatcgcagttgggaccagtgctctgcgcgcgcgagttgcggtacacggggttgcagcactggaacaccatcaaactggggtgcttcacgctggccagcacgctcttgtcgctgatctgatccttgtccaggtcctcggcgttgctcaggccgaacggggtcatcttgcacagctggcggcccaggaagggcacgctgtgaggcttgtggttacactcgcagtgcacgggcatcagcatcatccccgcgccgcgctgcatattcgggtagagggccttgacaaaggccaagatctgcttgaaagcttgctgggccttggccccctcgctgaagaacaggccgcagctcttcccgctgaactggttattcccacacccggcatcatgcacgcagcagcgcgcgtcatggctggtcagttgcaccacgcttcggccccagcggttctgggtcaccttggccttgctgggctgctccttcaacgcgcgctggccgttctcgctggtcacatccatctccaccacgtggtccttgtggatcatcaccgtcccgtgcagacacttgagctggccctcgacatcggtgcagccgtgttcccacagggcgcagccggtgcactcccaattcttgtgcgcgatcccgctgtggctgaagatgtaaccttgcaacatgcggcccatcacggtgctaaatgatttactggtgctgaaggtcagttgcaggccgcgggcctcctcgttcatccatgtctgacacattttctggaagatctcggtctgctcgggcatgagcttgtaggcatcgcgcaggccgctgtcgacgcggtagcgttccatcagcacgttcatggtatccatgcccttctcccaggacgagaccagaggcagactcagggggttgcgcacgttcaggataccgggggtcgcgggctcgacgatgcgttttccgtcctttccttccttcaacagaaccggcggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcggtccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctgaaacacctgcagcgccagtccctcatcctccgggacgccctagccgaccggagcgaaacccccctcagcgtcgaggagctgtgtcgggcctacgagctcaacctcttctcgccgcgcatgccccccaaacgccagcccaatggcacatgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctccttactctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagcttatcatgccccacatcgaggccctcgatgaaagtcaggagcagcgccccgaggacgcccggcccgtggtcagcgacgagatgctcgcgcgctggctcgggacccgcgacccccaggctttggaacagcggcgcaagctcatgctggccgtggtcctggtcaccctcgagctggaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctgggccagaccgtgctccactctaccctgaagggcgaggcgcggcgggactacgtccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgagacagacgctgaaaggcgggctgcccgacttcatgagccagagcatgttgcaaaactaccgcactttcattctcgagcgatctgggatgctgcccgccacctgcaacgccttcccctccgactttgtaccgctgagctaccgcgagtgtcccccgccgctgtggagccactgctacctcttgcagctggccaactacatcgcctaccactcggacgtgatcgaggacgtgagcggcgaggggctgctcgagtgccactgtcgctgcaacctgtgctccccgcatcgctccctggtctgcaacccccagctcctgagcgagacccaggtcatcggtaccttcgagctgcaaggtccgcaggagtccaccgctccgctgaaactcacgccggggttgtggacttccgcgtacctgcgcaaatttgtacccgaagactaccacgcccatgagataaagttctttgaggaccaatcgcgcccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagagcagcccgtcgccgcaccatccgcgccggcagccccgccggtcacggatacaacctccgctccggtcaagcctcctcgtagatgggatcaaatgaagggtgacggtaagcacgagcggcagggctaccgatcatggagggcccacaaagccgcgatcatcgcctgcttgcaagactgcggggggaacatcgcttttgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacaggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagcttaacgacgagttggtgagctcttcaatcggtctgcgaccggacggagtgttccaactagccggagccgggagatcctccttcactccccaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgatgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgttgtactctgtgtgctgagtttaataaaagctaaactcctacaatactctgggatcccgtgtcgtcgcactcgcaacgagatcttcaacctcaccaaccagactgaggtaaaactcaactgcagaccggggggcaaatacatcctctggctctttgaaaacacttccttcgcagtctccaacgcctgctgcactagtccatgaactgattgattaaagcccaaaaaaccaatcaaaccccttcccattagccccaaataaacaatcattggaaataatcattcaataaagatcacttacttgaaatctgaaagtatgtctctggtgtagttgtttagcagcacctcggtaccctcctcccagctctggtactccagtccccggcgggcggcgaactttctccacaccttgaaagggatgtcaaattcctggtccacaattttcattgttttccctctcagatgtcaaagaggcttcgggtggaagatgacttcaaccccgtctacccctatgactacgcgcggaatcagaatatccccttcctcactcccccctttgtctcctccgatggattccaaaacttcccccctggtgtcctgtcactcaaattggctgacccaatcgctatcaccaatggtgatgtctcactcaaggtgggagggggacttactttacaagatggaaccggaaaactaactatcgacaccaagactcccttgcaagttgcaaataataaattagaacttgcgtttgatgcaccattgtatgaaaaaaatggaaaacttgctttaaaaacaggccatggattagctgttttaaccaaagacattggcataccagaattaattggatcccttgtgatcttaactggaaaaggaattgggacaggtactgttgcaggaggaggaactatagatgtaagactgggtgatgatggaggtttatcatttgataaaaagggtgatctagtagcctggaataaaaaaaatgacaggcgcactttgtggacaacacctgatccatccccaaactgcagagtatcagaagataaagattcaaaactaactttaattcttacaaaatgtggaagtcagatcctagcaagtttttcactgcttgtagtcaaagggacgtatgcaactgttgataaaaatacaactaataaacaatttagcattaaactactgtttgatgcaaatggaaagcttaaaagcgaatcaaaccttagtggttattggaactatagaagtgataatagtgttgttagtactccctatgacaatgcagtgcctttcatgccaaataccacggcttatccaaaaatcattaatagtacaactgatcctgaaaataagaaaagttcggctaaaaaaactattgttggcaatgtctacctagaaggtaatgcgggtcaaccagtagccgttgctattagtttcaataaggaaactactgctgactattcaataacatttgactttgcgtggagcaaagcttatgaaacccctgtgccttttgacacctcctccatgacattctcatatattgcccaggaaaatcaagacaaaggcgaataaagtgttttgaaatgaacttatgtatctttattgatttttacaccagcacgggtagtcagtctcccaccaccagcccatttcacagtgtaaacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatttcgctgctgccgctcagtcatccaactgaagtacatccattctcgaagattctggagaagttcctctgcatctgatgaaataaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccgaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgtttttcgcgaaaacacttccgcgttctcacttcctcgtatcgatttcgtgacttaacttccgggttcccacgttacgtcacttctgcccttacatgtaactgagtcgtagggcgccatcttgcccacgtccaaaatggcttccatgtccagccacgcctccgcggcgaccgttagccgtgcgtcgtgacgtcatttgcatcgtcttctctcgtccaatcagcgtcggctccgccctaaattcaaaagctcatttgcatattaacttttgtttactttgtggggtatattattgatgatgttaat

SEQ ID NO: 9 - DNA, 30880 bp, Artificial Sequence
  Ad20-42-42.Luc+.5ORF6
attaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgcttttgagtaaactctcatccatctggtcagaaaagactatttttttattgtcaagcttggtggcgaaggagccatagagggcgttggagagaagcttggcgatggatctcatggtctgatttttgtcacggtcggcgcgctccttggccgcgatgtttagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtcgcccgcctttgcgcgaacagaaagggggcagcacatcaagcagatgctcgtcgggggggtccgcatcgatggtgaagataccgggacagagttccttgtcaaaatagtctatttttgaggatgcatcatccaaggccatctgccactcacgggcggccattgctcgctcgtaggggttgaggggcggaccccagggcatgggatgcgtgagggcggaggcgtacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtggggcaagcggaccgagtcgcggatgaagtgcgcgtaggagtcttgcagcttggcgacgagctcggcggtgacgaggacgtccatggcgcagtagtccagcgtttcgcggatgatgtcataacccgtctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctctagcgcccactcctggagatgtgggttggcttgcatgaaggaagcccagagctcgcgggccatgagggtctggagctcgtcacgaaagaggcggaactgctggcccacggccatcttttcgggtgtgacgcagtagaaggtgagggggtcccgctcccagcgatcccagcgtaagcgcacggcgagatcgcgagcgagggcgaccagctctgggtcccccgagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgtgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggtcccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcattcgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcgaagacggcgtagttgcgcaggcgctggaagaggtagttgagggtggtggcgatgtgctcggtgacgaagaagtacatgatccagcggcgcagaggcatctcgctgatgtcgccgatggcctccagcctttccatggcctcgtagaaatccacagcgaagttgaaaaactgggcgttgcgggccgagaccgtgagctcgtcttccaggagcctgatgagctcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagtgcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccaacgcccctgagcggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccatgagcgaccagttgacggtctgcaggccgggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctattcctccgtagcctggcggaacgcaaacgggttaggccgcgtgtgtaccccggttcgagtcccctcgaatcaggctggagccgcgactaacgtggtattggcactcccgtctcgacccgagcccgatagctgccaggatacggcggagagcccttttggcggccgaggggggtcgctagacttgaaagcggccgaaaaccctgccgggtagtggctcgcgcccgtagtctggagaagcatcgccagggttgagtcgcggcagaacccggttcgcggacggccgcggagagcgggacttggtcaccccgccgatttaaaaacccacagccagccgacttctccagttacgggagcgagcccccttttttctttttgccagatgcatcccgtcctgcgccaaatgcgtcccaccccccctccggcgaccaccgcaaccgcggccgtagcaggcgccggcgctagccagccacagccacagacagagatggacttggaagagggcgaagggctggcgagattgggggcgccgtctccggagcgacacccccgcgtgcagctgcagaaggacgtgcgcccggcgtacgtgcctgcgcagaacctgttcagggaccgcagcggggaggagcccgaggagatgcgcgactgccgctttcgggcgggcagggagctgcgcgagggtctggaccgccagcgcgtgctgcgcgatgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggtcgctggctgctggagctgatcaacatcttgcagagcatcgtagtgcaggagcgcagcctgagcctggccgagaaagtggcggcgatcaactactcggtgctgagcctgggcaagttttacgcgcgcaagatttacaagacgccatacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaagccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcggccattctggaggcggtggtcccctctcgaaccaaccccacgcacgagaaggtgctggcgatcgtgaacgcgctggcggagaacaaggccatccgtcccgacgaggccggtctggtgtacaacgccctgctggagcgcgtgggccgctacaacagcacaaacgtgcagtccaacctggaccggctggtgacggacgtgcgcgaggccgtggcgcagcgcgagcggttcaagaacgaaggcctgggctcgctggtggcgctgaacgccttcctggcgacgcagccggcgaacgtgccgcgcgggcaggacgattataccaactttataagcgcgctgcggctgatggtgaccgaggtgccccagagcgaggtgtaccagtcgggcccggattactttttccaaactagcagacagggcctgcagacggtgaacctgagccaggcctttaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcacaggtggacgagcagaccttccaggagatcactagcgtgagccgcgcgctggggcagaacgacaccgacagtcttagggccaccctgaacttcttgctgaccaatagacagcagaagatcccggcacagtacgcgctatcggccgaggaggagcgcatcctgagatatgtgcagcagagcgtagggctgttcctgatgcaggagggggccacccccagcgccgcgctggacatgaccgcgcgcaacatggaacctagcatgtacgctgccaaccggccgttcatcaataagctgatggactacctgcaccgcgcggcggccatgaactcggactactttacaaacgccatcctgaacccgcactggctcccgccgccggggttctacacgggcgagtacgatatgcccgaccccaacgacgggttcctgtgggacgacgtggacagcgcggtgttctccccgaccttgcaaaagcgccaggaggcggtgcgcacgcccgcgagcgagggtgcggtgggtcggagcccctttcctagcttagggagtttgcatagcttgccgggctcggtgaacagcggcagggtgagccgaccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccaaactggggcgacggatgttttgaaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttattcggagctggctccgctgtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcacccagagaatgtagctaaagacgatttgagtcaggataagcttgaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcagaaatttcaagctgggctgggacccggtgaccaagttggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaagatctagtagggggcaacatcccagccctcctgaatgtcaaggagtatctgaaggataaggaagaagctggcacagcagatgcaaataccattaaggctcagaatgatgcagtcccaagaggagataactatgcatcagcggcagaagccaaagcagcaggaaaagaaattgagttgaaggccattttgaaagatgattcaaacagaagctacaatgtgatcgagggaaccacagacaccctgtaccgcagttggtacctgtcctatacctacggggaccccgagaagggggtgcagtcgtggacgctgctcaccaccccggacgtcacatgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctatccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttctaccgccgctgccgtgcgtaccaccgtcgacgacgtcatcgactcggtggtcgccgacgcgcgcaactacacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcctgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccaccaacgtgcccatctccatcccctcgcgcaactgggccgccttccgcggctggagtttcacccggctcaagaccaaggaaactccctcccttggctcgggttttgacccctactttgtctactcgggctccatcccctacctcgacgggaccttttacctgaaccataccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttccttgtccagatgctctcccactacaacatcggctaccagggcttccacgtgcccgagggctacaaggaccgcatgtactcattcttccgcaacttccagcccatgagcaggcaggtggtcgatgagatcaactacaaggactacaaagccgtcaccctgcccttccagcacaacaactcgggcttcaccggctatctcgcccccaccatgcgccaggggcagccctaccccgccaacttcccatacccgctcatcggccagacagccgtaccctccgtcacccagaaaaagttcctctgcgacagggtcatgtggcgcatccccttttccagcaacttcatgtccatgggcgccctcaccgacctgggacagaacatgctctatgccaactcggcccacgcgctcgacataacttttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctactacataagcatgagcggctccagcgaacgagagctcgcggccatcgtgcgcgacctgggctgcgggccctactttttgggaacccacgacaagcgcttccctggcttccttgccagcgacaagctggcatgcgccatcgtcaacacggccggccgcgagaccgggggcgtgcactggctcgcctttggctggaatccgcgctcgcgcacctgctacatgtttgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttttgttgcatgtttttgcatgcctttgtgcactggcccgaccgacccatggacggaaaccccaccatgaacttgctgacgggggtgcccaacggcatgctacaatcgccacaggtgctccccaccctcaggcgcaaccaggaggagctctaccgcttcctcgcgcgccactccccttactttcgatcccaccgcgccgccattgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactgaaacttgggctgccacttgaactcggggatcaccagtttgggcactggggtctcggggaaggtctcgctccacatgcgccggctcatctgcagggcgcccagtatgtcgggcgcggagatcttgaaatcgcagttgggaccagtgctctgcgcgcgcgagttgcggtacacggggttgcagcactggaacaccatcaaactggggtgcttcacgctggccagcacgctcttgtcgctgatctgatccttgtccaggtcctcggcgttgctcaggccgaacggggtcatcttgcacagctggcggcccaggaagggcacgctgtgaggcttgtggttacactcgcagtgcacgggcatcagcatcatccccgcgccgcgctgcatattcgggtagagggccttgacaaaggccaagatctgcttgaaagcttgctgggccttggccccctcgctgaagaacaggccgcagctcttcccgctgaactggttattcccacacccggcatcatgcacgcagcagcgcgcgtcatggctggtcagttgcaccacgcttcggccccagcggttctgggtcaccttggccttgctgggctgctccttcaacgcgcgctggccgttctcgctggtcacatccatctccaccacgtggtccttgtggatcatcaccgtcccgtgcagacacttgagctggccctcgacatcggtgcagccgtgttcccacagggcgcagccggtgcactcccaattcttgtgcgcgatcccgctgtggctgaagatgtaaccttgcaacatgcggcccatcacggtgctaaatgatttactggtgctgaaggtcagttgcaggccgcgggcctcctcgttcatccatgtctgacacattttctggaagatctcggtctgctcgggcatgagcttgtaggcatcgcgcaggccgctgtcgacgcggtagcgttccatcagcacgttcatggtatccatgcccttctcccaggacgagaccagaggcagactcagggggttgcgcacgttcaggataccgggggtcgcgggctcgacgatgcgttttccgtcctttccttccttcaacagaaccggcggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcggtccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctgaaacacctgcagcgccagtccctcatcctccgggacgccctagccgaccggagcgaaacccccctcagcgtcgaggagctgtgtcgggcctacgagctcaacctcttctcgccgcgcatgccccccaaacgccagcccaatggcacatgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctccttactctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagcttatcatgccccacatcgaggccctcgatgaaagtcaggagcagcgccccgaggacgcccggcccgtggtcagcgacgagatgctcgcgcgctggctcgggacccgcgacccccaggctttggaacagcggcgcaagctcatgctggccgtggtcctggtcaccctcgagctggaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctgggccagaccgtgctccactctaccctgaagggcgaggcgcggcgggactacgtccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgagacagacgctgaaaggcgggctgcccgacttcatgagccagagcatgttgcaaaactaccgcactttcattctcgagcgatctgggatgctgcccgccacctgcaacgccttcccctccgactttgtaccgctgagctaccgcgagtgtcccccgccgctgtggagccactgctacctcttgcagctggccaactacatcgcctaccactcggacgtgatcgaggacgtgagcggcgaggggctgctcgagtgccactgtcgctgcaacctgtgctccccgcatcgctccctggtctgcaacccccagctcctgagcgagacccaggtcatcggtaccttcgagctgcaaggtccgcaggagtccaccgctccgctgaaactcacgccggggttgtggacttccgcgtacctgcgcaaatttgtacccgaagactaccacgcccatgagataaagttctttgaggaccaatcgcgcccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagagcagcccgtcgccgcaccatccgcgccggcagccccgccggtcacggatacaacctccgctccggtcaagcctcctcgtagatgggatcaaatgaagggtgacggtaagcacgagcggcagggctaccgatcatggagggcccacaaagccgcgatcatcgcctgcttgcaagactgcggggggaacatcgcttttgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacaggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagcttaacgacgagttggtgagctcttcaatcggtctgcgaccggacggagtgttccaactagccggagccgggagatcctccttcactccccaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgatgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgttgtactctgtgtgctgagtttaataaaagctaaactcctacaatactctgggatcccgtgtcgtcgcactcgcaacgagatcttcaacctcaccaaccagactgaggtaaaactcaactgcagaccggggggcaaatacatcctctggctctttgaaaacacttccttcgcagtctccaacgcctgctgcactagtccatgaactgattgattaaagcccaaaaaaccaatcaaaccccttcccattagccccaaataaacaatcattggaaataatcattcaataaagatcacttacttgaaatctgaaagtatgtctctggtgtagttgtttagcagcacctcggtaccctcctcccagctctggtactccagtccccggcgggcggcgaactttctccacaccttgaaagggatgtcaaattcctggtccacaattttcattgttttccctctcagatgtcaaagaggcttcgggtggaagatgacttcaaccccgtctacccctatgactacgcgcggaatcagaatatccccttcctcactcccccctttgtctcctccgatggattccaaaacttcccccctggtgtcctgtcactcaaattggctgacccaatcgctatcaccaatggtgatgtctcactcaaggtgggagggggacttactttacaagatggaaccggaaaactaactatcgacaccaagactcccttgcaagttgcaaataataaattagaacttgcgtttgatgcaccattgtatgaaaaaaatggaaaacttgctttaaaaacaggccatggattagctgttttaaccaaagacattggcataccagaattaattggatcccttgtgatcttaactggaaaaggaattgggacaggtactgttgcaggaggaggaactatagatgtaagactgggtgatgatggaggtttatcatttgataaaaagggtgatctagtagcctggaataaaaaaaatgacaggcgcactttgtggacaacacctgatccatccccaaactgcagagtatcagaagataaagattcaaaactaactttaattcttacaaaatgtggaagtcagatcctagcaagtttttcactgcttgtagtcaaagggacgtatgcaactgttgataaaaatacaactaataaacaatttagcattaaactactgtttgatgcaaatggaaagcttaaaagcgaatcaaaccttagtggttattggaactatagaagtgataatagtgttgttagtactccctatgacaatgcagtgcctttcatgccaaataccacggcttatccaaaaatcattaatagtacaactgatcctgaaaataagaaaagttcggctaaaaaaactattgttggcaatgtctacctagaaggtaatgcgggtcaaccagtagccgttgctattagtttcaataaggaaactactgctgactattcaataacatttgactttgcgtggagcaaagcttatgaaacccctgtgccttttgacacctcctccatgacattctcatatattgcccaggaaaatcaagacaaaggcgaataaagtgttttgaaatgaacttatgtatctttattgatttttacaccagcacgggtagtcagtctcccaccaccagcccatttcacagtgtaaacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatttcgctgctgccgctcagtcatccaactgaagtacatccattctcgaagattctggagaagttcctctgcatctgatgaaataaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccgaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgtttttcgcgaaaacacttccgcgttctcacttcctcgtatcgatttcgtgacttaacttccgggttcccacgttacgtcacttctgcccttacatgtaactgagtcgtagggcgccatcttgcccacgtccaaaatggcttccatgtccagccacgcctccgcggcgaccgttagccgtgcgtcgtgacgtcatttgcatcgtcttctctcgtccaatcagcgtcggctccgccctaaattcaaaagctcatttgcatattaacttttgtttactttgtggggtatattattgatgatgttaat

SEQ ID NO: 10 - DNA, 29981 bp, Artificial Sequence
  Ad20-42-42.eGFP.5ORF6
attaacatcatcaataatataccccacaaagtaaacaaaagttaatatgcaaatgagcttttgaatttagggcggagccgacgctgattggacgagagaagacgatgcaaatgacgtcacgacgcacggctaacggtcgccgcggaggcgtggcctagcccggaagcaagtcgcggggctgatgacgtataaaaaagcggactttagacccggaaatggccgattttcccgcggccacggccggatatgaggtaattctgggcggatgcaagtgaaattaggtcattttggcgcgaaaactgaatgaggaagtgaaaagcgaaaaataccgatcccgcccagggcggaatatttaccgagggccgagagactttgaccgattacgtgggggtttcgattgcggtgtttttttcgcgaatttccgcgtccgtgtcaaagtccggtgtttatgtcacagatcagctgacctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagcttggtaccggtgaattcgctagcgttaacggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagacgagatccgaacttgtttattgcagcttataatggttacaaataaagcaatagcatcacaaatttcacaaataaagcatttttttcactgcattctagttgtggtttgtccaaactcatcaatgtatcttatcatgtctcgatccaggtaggtttgagtagtgggcgtggctaagatgactataaaggtgggtgtcttacgagggtctttttgcttttctgcagacatcatgaacgggaccggcggggccttcgaaggggggctttttagcccttatttgacaacccgcctgccgggatgggccggagttcgtcagaatgtgatgggatcgacggtggacgggcgtccagtgcttccagcaaattcctcgaccatgacctacgcgaccgtggggaactcgtcgctcgacagcaccgccgcagccgcggcagccgcagccgccatgacagcgacgagactggcttcgagctacatgcccagcagcggtagcagcccctctgtgcccagttccatcatcgccgaggagaaactgctggccctcctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagagatgggattggatgttgaggtacatgggcatgagcccgtcccgggggtgaaggtagcaccactgcatggcctcatgctctggggtcgtgttgtagatgatccagtcatagcaggggcgctgggcgtggtgctggatgatgtctttgaggaggagactgatggccacggggagccccttggtgtaggtgttggcgaagcggttgagctgggagggatgcatgcggggggagatgatgtgcagtttggcctggatcttgaggttggcaatgttgccgcccagatcccgccgggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcataattatgctcctgggtgagatcatcataagacattttaatgaatttggggcggagggtgccagattgggggacgatcgttccctcgggccccggggcgaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggagatgcagctgccgtcttcccggaggaggggggccacctcgttgagcatgtctctgacttggaggttttcccggacgagctcgccgaggaggcggtccccgcccagcgagagcagctcttgcagggaagcaaagtttttcaggggcttgagcccgtcggccatgggcatcttggcgagggtctgcgagaggagttccaagcggtcccagagctcggtgacgtgctctacggcatctcgatccaacagacttcctcgtttcgggggttgggacgactgcgactgtagggcacgagacgatgggcgtccagcgctgccagcgtcatgtccttccagggtctcagggtccgcgtgagcgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaagcgtccgctccgcagtgggcgcagacggtctcgcactcgacgagccaggtgagctcgggatgttcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcggaccactctgagacgaaggcgcgcgtccacgccaagacaaaggaggccacgtgcgaggggtagcggtcgttgtccaccagggggtccaccttttccaccgtgtgcagacacatgtccccctcctcagcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgcttttgagtaaactctcatccatctggtcagaaaagactatttttttattgtcaagcttggtggcgaaggagccatagagggcgttggagagaagcttggcgatggatctcatggtctgatttttgtcacggtcggcgcgctccttggccgcgatgtttagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtcgcccgcctttgcgcgaacagaaagggggcagcacatcaagcagatgctcgtcgggggggtccgcatcgatggtgaagataccgggacagagttccttgtcaaaatagtctatttttgaggatgcatcatccaaggccatctgccactcacgggcggccattgctcgctcgtaggggttgaggggcggaccccagggcatgggatgcgtgagggcggaggcgtacatgccgcagatgtcatagacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtggggcaagcggaccgagtcgcggatgaagtgcgcgtaggagtcttgcagcttggcgacgagctcggcggtgacgaggacgtccatggcgcagtagtccagcgtttcgcggatgatgtcataacccgtctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtccctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcgaagccgttgatgttgtgcccgacgatgtagagttccatgaatcgcgggcggcctttgatgtgcggcagctttttgagctcctcgtaggtgaggtcctcggggcattgcaggccgtgctgctctagcgcccactcctggagatgtgggttggcttgcatgaaggaagcccagagctcgcgggccatgagggtctggagctcgtcacgaaagaggcggaactgctggcccacggccatcttttcgggtgtgacgcagtagaaggtgagggggtcccgctcccagcgatcccagcgtaagcgcacggcgagatcgcgagcgagggcgaccagctctgggtcccccgagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgtgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggtcccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctcgacggtggcggcgaggtcattcgagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcgaagacggcgtagttgcgcaggcgctggaagaggtagttgagggtggtggcgatgtgctcggtgacgaagaagtacatgatccagcggcgcagaggcatctcgctgatgtcgccgatggcctccagcctttccatggcctcgtagaaatccacagcgaagttgaaaaactgggcgttgcgggccgagaccgtgagctcgtcttccaggagcctgatgagctcggcgatggtggcgcgcacctcgcgctcgaaatccccgggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagtgcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccaacgcccctgagcggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccatgagcgaccagttgacggtctgcaggccgggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctattcctccgtagcctggcggaacgcaaacgggttaggccgcgtgtgtaccccggttcgagtcccctcgaatcaggctggagccgcgactaacgtggtattggcactcccgtctcgacccgagcccgatagctgccaggatacggcggagagcccttttggcggccgaggggggtcgctagacttgaaagcggccgaaaaccctgccgggtagtggctcgcgcccgtagtctggagaagcatcgccagggttgagtcgcggcagaacccggttcgcggacggccgcggagagcgggacttggtcaccccgccgatttaaaaacccacagccagccgacttctccagttacgggagcgagcccccttttttctttttgccagatgcatcccgtcctgcgccaaatgcgtcccaccccccctccggcgaccaccgcaaccgcggccgtagcaggcgccggcgctagccagccacagccacagacagagatggacttggaagagggcgaagggctggcgagattgggggcgccgtctccggagcgacacccccgcgtgcagctgcagaaggacgtgcgcccggcgtacgtgcctgcgcagaacctgttcagggaccgcagcggggaggagcccgaggagatgcgcgactgccgctttcgggcgggcagggagctgcgcgagggtctggaccgccagcgcgtgctgcgcgatgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatcgcgcgcgaggaggtggccctgggcctgatgcacctgtgggacctggcggaggccatcgtgcagaacccggacagcaagcctctgacggcgcagctgttcctggtggtgcagcacagtagggacaacgaggcgttcagggaggcgctgctgaacatcgccgagcccgagggtcgctggctgctggagctgatcaacatcttgcagagcatcgtagtgcaggagcgcagcctgagcctggccgagaaagtggcggcgatcaactactcggtgctgagcctgggcaagttttacgcgcgcaagatttacaagacgccatacgtgcccatagacaaggaggtgaagatagacagcttttacatgcgcatggcgctcaaggtgctgacgctgagcgacgacctgggcgtgtaccgcaacgaccgcatccacaaggccgtgagcgcgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaagccgcctacggtccagaggacttggatgaggatgaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcggccattctggaggcggtggtcccctctcgaaccaaccccacgcacgagaaggtgctggcgatcgtgaacgcgctggcggagaacaaggccatccgtcccgacgaggccggtctggtgtacaacgccctgctggagcgcgtgggccgctacaacagcacaaacgtgcagtccaacctggaccggctggtgacggacgtgcgcgaggccgtggcgcagcgcgagcggttcaagaacgaaggcctgggctcgctggtggcgctgaacgccttcctggcgacgcagccggcgaacgtgccgcgcgggcaggacgattataccaactttataagcgcgctgcggctgatggtgaccgaggtgccccagagcgaggtgtaccagtcgggcccggattactttttccaaactagcagacagggcctgcagacggtgaacctgagccaggcctttaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcacaggtggacgagcagaccttccaggagatcactagcgtgagccgcgcgctggggcagaacgacaccgacagtcttagggccaccctgaacttcttgctgaccaatagacagcagaagatcccggcacagtacgcgctatcggccgaggaggagcgcatcctgagatatgtgcagcagagcgtagggctgttcctgatgcaggagggggccacccccagcgccgcgctggacatgaccgcgcgcaacatggaacctagcatgtacgctgccaaccggccgttcatcaataagctgatggactacctgcaccgcgcggcggccatgaactcggactactttacaaacgccatcctgaacccgcactggctcccgccgccggggttctacacgggcgagtacgatatgcccgaccccaacgacgggttcctgtgggacgacgtggacagcgcggtgttctccccgaccttgcaaaagcgccaggaggcggtgcgcacgcccgcgagcgagggtgcggtgggtcggagcccctttcctagcttagggagtttgcatagcttgccgggctcggtgaacagcggcagggtgagccgaccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccaaactggggcgacggatgttttgaaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttattcggagctggctccgctgtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcacccagagaatgtagctaaagacgatttgagtcaggataagcttgaatatgagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcagaaatttcaagctgggctgggacccggtgaccaagttggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaagatctagtagggggcaacatcccagccctcctgaatgtcaaggagtatctgaaggataaggaagaagctggcacagcagatgcaaataccattaaggctcagaatgatgcagtcccaagaggagataactatgcatcagcggcagaagccaaagcagcaggaaaagaaattgagttgaaggccattttgaaagatgattcaaacagaagctacaatgtgatcgagggaaccacagacaccctgtaccgcagttggtacctgtcctatacctacggggaccccgagaagggggtgcagtcgtggacgctgctcaccaccccggacgtcacatgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctatccagtcgcaccttctaaaaaatgtctattctcatctcgcccagcaataacaccggctggggtattactaggcccagcaccatgtacggaggagccaagaagcgctcccagcagcaccccgtccgcgtccgcggccacttccgcgctccctggggcgcttacaagcgcgggcggacttctaccgccgctgccgtgcgtaccaccgtcgacgacgtcatcgactcggtggtcgccgacgcgcgcaactacacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacaggccgccgggccatgatgcgagccgcgcgccgcgctgccactgcacccacccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaacccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcctggcgagcggtcctcggtcaggagcaagcgtagctatgacgaggtatacggcgacgacgacatcctggaccaggcggcggagcgggcgggcgagtttgcctacgggaagcggtcgcgcgaagaggagctgatctcgctgccgctggacgaaagcaaccccacgccaagcctgaagcccgtgaccctgcagcaggtgctgccccaggcggtgctgctgccgagccgcggggtcaagcgcgagggcgagaacatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccactgctaccgcctccacttctaccgtcgccacggccaccgagcctcccaggaggcgaagatggggccctgccaaccggctgatgcccaactacgtgttgcatccttccatcatcccgacgccgggctaccgcggcactcggtactacgccagccgcaggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctacccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcgcccgcccacggtggtggagatggaagatgcaactcttccgccgcccaaaggcgagaagcggccgcggcccgacgcggaggagacgatcctgcaggtggacgagccgccctcgtacgaggaggccgtgaaggccggcatgcccaccacgcgcatcatcgcgccgctggccacgggtgtaatgaaacccgccacccttgacctgcctccaccacccacgcccgctccaccgaaggcagctccggttgtgcagccccctcctgtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcctacaacagtctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatacaacagaaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacaatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgctttccattggacggctcaggcactaattcagcttttcaaggtgtaaaaattaagcaaaaccaagacggagatgttaatgatgattgggaaaaggatgataaggtatccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccgccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttcccttggaaacgacctgcgcgttgacggcgcctccgtccgcttcgacagtgttaacctctatgccaccttcttccccatggcgcacaacaccgcctccaccctggaagccatgctgcgcaacgacaccaacgaccagtccttcaacgactacctctcggccgccaacatgctctaccctatcccggccaaggccaccaacgtgcccatctccatcccctcgcgcaactgggccgccttccgcggctggagtttcacccggctcaagaccaaggaaactccctcccttggctcgggttttgacccctactttgtctactcgggctccatcccctacctcgacgggaccttttacctgaaccataccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttccttgtccagatgctctcccactacaacatcggctaccagggcttccacgtgcccgagggctacaaggaccgcatgtactcattcttccgcaacttccagcccatgagcaggcaggtggtcgatgagatcaactacaaggactacaaagccgtcaccctgcccttccagcacaacaactcgggcttcaccggctatctcgcccccaccatgcgccaggggcagccctaccccgccaacttcccatacccgctcatcggccagacagccgtaccctccgtcacccagaaaaagttcctctgcgacagggtcatgtggcgcatccccttttccagcaacttcatgtccatgggcgccctcaccgacctgggacagaacatgctctatgccaactcggcccacgcgctcgacataacttttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctactacataagcatgagcggctccagcgaacgagagctcgcggccatcgtgcgcgacctgggctgcgggccctactttttgggaacccacgacaagcgcttccctggcttccttgccagcgacaagctggcatgcgccatcgtcaacacggccggccgcgagaccgggggcgtgcactggctcgcctttggctggaatccgcgctcgcgcacctgctacatgtttgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttttgttgcatgtttttgcatgcctttgtgcactggcccgaccgacccatggacggaaaccccaccatgaacttgctgacgggggtgcccaacggcatgctacaatcgccacaggtgctccccaccctcaggcgcaaccaggaggagctctaccgcttcctcgcgcgccactccccttactttcgatcccaccgcgccgccattgaaaacgccaccgcttttgataaaatgaaacaactgcgtgtatctcaataaacagcactttattttacatgcactggagtatatgcaagttatttaaaagtcgaaggggttctcgcgctcgtcgttgtgcgccgcgctggggagggccacgttgcggtactgaaacttgggctgccacttgaactcggggatcaccagtttgggcactggggtctcggggaaggtctcgctccacatgcgccggctcatctgcagggcgcccagtatgtcgggcgcggagatcttgaaatcgcagttgggaccagtgctctgcgcgcgcgagttgcggtacacggggttgcagcactggaacaccatcaaactggggtgcttcacgctggccagcacgctcttgtcgctgatctgatccttgtccaggtcctcggcgttgctcaggccgaacggggtcatcttgcacagctggcggcccaggaagggcacgctgtgaggcttgtggttacactcgcagtgcacgggcatcagcatcatccccgcgccgcgctgcatattcgggtagagggccttgacaaaggccaagatctgcttgaaagcttgctgggccttggccccctcgctgaagaacaggccgcagctcttcccgctgaactggttattcccacacccggcatcatgcacgcagcagcgcgcgtcatggctggtcagttgcaccacgcttcggccccagcggttctgggtcaccttggccttgctgggctgctccttcaacgcgcgctggccgttctcgctggtcacatccatctccaccacgtggtccttgtggatcatcaccgtcccgtgcagacacttgagctggccctcgacatcggtgcagccgtgttcccacagggcgcagccggtgcactcccaattcttgtgcgcgatcccgctgtggctgaagatgtaaccttgcaacatgcggcccatcacggtgctaaatgatttactggtgctgaaggtcagttgcaggccgcgggcctcctcgttcatccatgtctgacacattttctggaagatctcggtctgctcgggcatgagcttgtaggcatcgcgcaggccgctgtcgacgcggtagcgttccatcagcacgttcatggtatccatgcccttctcccaggacgagaccagaggcagactcagggggttgcgcacgttcaggataccgggggtcgcgggctcgacgatgcgttttccgtcctttccttccttcaacagaaccggcggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcggtccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctccagcatggctacctgggaggagaggaggatgtgctgctgaaacacctgcagcgccagtccctcatcctccgggacgccctagccgaccggagcgaaacccccctcagcgtcgaggagctgtgtcgggcctacgagctcaacctcttctcgccgcgcatgccccccaaacgccagcccaatggcacatgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctccttactctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagcttatcatgccccacatcgaggccctcgatgaaagtcaggagcagcgccccgaggacgcccggcccgtggtcagcgacgagatgctcgcgcgctggctcgggacccgcgacccccaggctttggaacagcggcgcaagctcatgctggccgtggtcctggtcaccctcgagctggaatgcatgcgccgcttcttcagcgaccccgagaccctgcgcaaggtcgaggagaccctgcactacactttcaggcacggtttcgtcaggcaggcctgcaagatctccaacgtggagctgaccaacctggtctcctgcctggggatcctgcacgagaaccgcctgggccagaccgtgctccactctaccctgaagggcgaggcgcggcgggactacgtccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgagacagacgctgaaaggcgggctgcccgacttcatgagccagagcatgttgcaaaactaccgcactttcattctcgagcgatctgggatgctgcccgccacctgcaacgccttcccctccgactttgtaccgctgagctaccgcgagtgtcccccgccgctgtggagccactgctacctcttgcagctggccaactacatcgcctaccactcggacgtgatcgaggacgtgagcggcgaggggctgctcgagtgccactgtcgctgcaacctgtgctccccgcatcgctccctggtctgcaacccccagctcctgagcgagacccaggtcatcggtaccttcgagctgcaaggtccgcaggagtccaccgctccgctgaaactcacgccggggttgtggacttccgcgtacctgcgcaaatttgtacccgaagactaccacgcccatgagataaagttctttgaggaccaatcgcgcccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagagcagcccgtcgccgcaccatccgcgccggcagccccgccggtcacggatacaacctccgctccggtcaagcctcctcgtagatgggatcaaatgaagggtgacggtaagcacgagcggcagggctaccgatcatggagggcccacaaagccgcgatcatcgcctgcttgcaagactgcggggggaacatcgcttttgcccgccgctacctgctcttccaccgcggggtgaacatcccccgcaacgtgttgcattactaccgtcaccttcacagctaagaaaaagcaagtaagaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacaggtcaacggggtccgtaaccatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagcttaacgacgagttggtgagctcttcaatcggtctgcgaccggacggagtgttccaactagccggagccgggagatcctccttcactccccaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgatgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtcctaccccgaagttgcatgagaatttttgttgtactctgtgtgctgagtttaataaaagctaaactcctacaatactctgggatcccgtgtcgtcgcactcgcaacgagatcttcaacctcaccaaccagactgaggtaaaactcaactgcagaccggggggcaaatacatcctctggctctttgaaaacacttccttcgcagtctccaacgcctgctgcactagtccatgaactgattgattaaagcccaaaaaaccaatcaaaccccttcccattagccccaaataaacaatcattggaaataatcattcaataaagatcacttacttgaaatctgaaagtatgtctctggtgtagttgtttagcagcacctcggtaccctcctcccagctctggtactccagtccccggcgggcggcgaactttctccacaccttgaaagggatgtcaaattcctggtccacaattttcattgttttccctctcagatgtcaaagaggcttcgggtggaagatgacttcaaccccgtctacccctatgactacgcgcggaatcagaatatccccttcctcactcccccctttgtctcctccgatggattccaaaacttcccccctggtgtcctgtcactcaaattggctgacccaatcgctatcaccaatggtgatgtctcactcaaggtgggagggggacttactttacaagatggaaccggaaaactaactatcgacaccaagactcccttgcaagttgcaaataataaattagaacttgcgtttgatgcaccattgtatgaaaaaaatggaaaacttgctttaaaaacaggccatggattagctgttttaaccaaagacattggcataccagaattaattggatcccttgtgatcttaactggaaaaggaattgggacaggtactgttgcaggaggaggaactatagatgtaagactgggtgatgatggaggtttatcatttgataaaaagggtgatctagtagcctggaataaaaaaaatgacaggcgcactttgtggacaacacctgatccatccccaaactgcagagtatcagaagataaagattcaaaactaactttaattcttacaaaatgtggaagtcagatcctagcaagtttttcactgcttgtagtcaaagggacgtatgcaactgttgataaaaatacaactaataaacaatttagcattaaactactgtttgatgcaaatggaaagcttaaaagcgaatcaaaccttagtggttattggaactatagaagtgataatagtgttgttagtactccctatgacaatgcagtgcctttcatgccaaataccacggcttatccaaaaatcattaatagtacaactgatcctgaaaataagaaaagttcggctaaaaaaactattgttggcaatgtctacctagaaggtaatgcgggtcaaccagtagccgttgctattagtttcaataaggaaactactgctgactattcaataacatttgactttgcgtggagcaaagcttatgaaacccctgtgccttttgacacctcctccatgacattctcatatattgcccaggaaaatcaagacaaaggcgaataaagtgttttgaaatgaacttatgtatctttattgatttttacaccagcacgggtagtcagtctcccaccaccagcccatttcacagtgtaaacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatttcgctgctgccgctcagtcatccaactgaagtacatccattctcgaagattctggagaagttcctctgcatctgatgaaataaaaaacccgtccatgcgaattcccctcatcacatcagccaggactctgtaggccatccccatccagttaatgctgccttgtctatcattcagagggggcggtggcaggattggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgtacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccgaagctagcacccaaaaactgcatgctggaataagctctctttgtgtctccggtgatgccttccaaaatgtgagtgataaagcgtggtagtttttctttaatcatttgcgtaatagaaaagtcctgtaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcaacagggtacccactgggtggcccttgcggacatacaaaaatcggtccgtgtgattaaaaagcagcacagtaagttcctgtcttcttccggcaaaaatcacatcggactgggttagtatgtccctggcatggtagtcattcaaggccataaatctgccctgatatccagtaggaaccagcacactcacttttaggtgaagcaataccaccccatgcggaggaatgtggaaagattcagggcaaaaaaaattatatctattgctagtcccttcctggacgggagcaatccctccaggactatctatgaaagcatacagagattcagccatagctcagcccgcttaccagtagacagagagcacagcagtacaagcgccaacagcagcgactgactacccactgacccagctccctatttaaaggcgccttacactgacgtaatgaccaaaggtctaaaaaccccgccaaaaaaaaacacacacgccctgggtgtttttcgcgaaaacacttccgcgttctcacttcctcgtatcgatttcgtgacttaacttccgggttcccacgttacgtcacttctgcccttacatgtaactgagtcgtagggcgccatcttgcccacgtccaaaatggcttccatgtccagccacgcctccgcggcgaccgttagccgtgcgtcgtgacgtcatttgcatcgtcttctctcgtccaatcagcgtcggctccgccctaaattcaaaagctcatttgcatattaacttttgtttactttgtggggtatattattgatgatgttaat

SEQ ID NO: 11 - protein, 371 aa, Artificial Sequence
  AdV-42 Fiber
MSKRLRVEDDFNPVYPYDYARNQNIPFLTPPFVSSDGFQNFPPGVLSLKLADPIAITNGDVSLKVGGGLTLQDGTGKLTIDTKTPLQVANNKLELAFDAPLYEKNGKLALKTGHGLAVLTKDIGIPELIGSLVILTGKGIGTGTVAGGGTIDVRLGDDGGLSFDKKGDLVAWNKKNDRRTLWTTPDPSPNCRVSEDKDSKLTLILTKCGSQILASFSLLVVKGTYATVDKNTTNKQFSIKLLFDANGKLKSESNLSGYWNYRSDNSVVSTPYDNAVPFMPNTTAYPKIINSTTDPENKKSSAKKTIVGNVYLEGNAGQPVAVAISFNKETTADYSITFDFAWSKAYETPVPFDTSSMTFSYIAQENQDKGE

SEQ ID NO: 12 - protein, 660 aa, Artificial Sequence
  AdV-42 hexon
MCLTARERAKMATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWADKERVNGGGNTKDVTKTFGVAAMGGEDITEKGLKIGTDTTENEPIFADKNFQPEPQVGEENWQETFVFYGGRALKKETKMKPCYGSFARPTNEKGGQAKFIIGDNGQPTENHDITMAFFDTPGGTITGGTGGPQDELKADIVMYTENINLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSAFQGVKIKQNQDGDVNDDWEKDDKVSTQNQICKGNIYAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLY

SEQ ID NO: 13 - protein, 519 aa, Artificial Sequence
  AdV-20 penton
MRRAVVSSSPPPSYESVMAQATLEVPFVPPRYMAPTEGRNSIRYSELAPLYDTTRVYLVDNKSADIASLNYQNDHSNFLTTVVQNNDFTPAEASTQTINFDERSRWGGDLKTILHTNMPNVNEYMFTSKFKARVMVARKHPENVAKDDLSQDKLEYEWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLVGGNIPALLNVKEYLKDKEEAGTADANTIKAQNDAVPRGDNYASAAEAKAAGKEIELKAILKDDSNRSYNVIEGTTDTLYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

(19) United States
(12) Patent Application Publication
(10) Pub. No.: US 2022/0372515 A1
(43) Pub. Date: Nov. 24, 2022

(54) ADENOVIRUS VECTORS AND USES THEREOF

(71) Applicants: Janssen Vaccines & Prevention B.V., Leiden (NL); Batavia Biosciences B.V., Leiden (NL); The University Court of the University of Edinburgh, Edinburgh (GB); Niklas Arnberg Konsult AB, Umea (SE)

(72) Inventors: Jerome Hubertina Henricus Victor CUSTERS, Leiden (NL); Menzo Jans Emko HAVENGA, Leiden (NL); Monika BALLMANN, Leiden (NL); Angelique Alida Corina LEMCKERT, Leiden (NL); Andrew BAKER, Edinburgh (GB); Niklas ARNBERG, Umea (SE); Gyozo KAJAN, Budapest (HU)

(21) Appl. No.: 17/761,058

(22) PCT Filed: Oct. 2, 2020
(86) PCT No.: PCT/IB2020/059289

§ 371 Date: Mar. 16, 2022

Related U.S. Application Data
(60) Provisional application No. 62/909,853, filed on Oct. 3, 2019.

(51) Int. Cl. C12N 15/86; C07K 14/005

(52) U.S. Cl. CPC C12N 15/86; C07K 14/005; C12N 2710/10322; C12N 2710/10343; C12N 2710/10352; A61K 39/00

(57) ABSTRACT

Provided herein are chimeric adenoviral vectors. The provided chimeric adenoviral vectors can be used to induce a protective immune response and/or express a transgene in a subject in need thereof.

### CROSS REFERENCE TO RELATED APPLICATION

This application claims priority to U.S. Provisional Application 62/909,853, filed Oct. 3, 2019, the disclosure of which is incorporated herein by reference in its entirety.

### FIELD OF THE INVENTION

This invention relates to biotechnology. More particularly, to the field and use of adenoviral vectors, such as replication deficient adenoviral vectors to deliver antigens and elicit an immune response in hosts.

### REFERENCE TO SEQUENCE LISTING SUBMITTED ELECTRONICALLY

This application contains a sequence listing, which is submitted electronically via EFS-Web as an ASCII formatted sequence listing with a file name “004852.120WO1 Sequence Listing” and a creation date of Aug. 13, 2020 and having a size of 262 kb. The sequence listing submitted via EFS-Web is part of the specification and is herein incorporated by reference in its entirety.

### BACKGROUND OF THE INVENTION

Recombinant adenoviral vectors are widely applied for gene therapy applications and vaccines. AdV-5 vector-based vaccines have been shown to elicit potent and protective immune responses in a variety of animal models (see, e.g., WO2001/02607; WO2002/22080; Shiver et al., Nature 415:331 (2002); Letvin et al., Ann. Rev. Immunol. 20:73 (2002); Shiver and Emini, Ann. Rev. Med. 55:355 (2004)). However, the utility of recombinant AdV-5 vector-based vaccines will likely be limited by the high seroprevalence of AdV-5-specific neutralizing antibodies

(NAbs) in human populations. The existence of anti-AdV-5 immunity has been shown to substantially suppress the immunogenicity of AdV-5-based vaccines in studies in mice, rhesus monkeys, and humans.

One promising strategy to circumvent the existence of pre-existing immunity in individuals previously infected or treated with the most common human adenovirus, e.g., AdV-5, involves the development of recombinant vectors from adenovirus serotypes that do not encounter such pre-existing immunities. One such strategy is based on the use of chimeric adenoviruses comprising replacement of native capsid protein sequences (e.g., hexon and/or fiber protein sequences) with capsid protein sequences (e.g., hexon and/or fiber protein sequences) from adenoviruses with low (or no) seroprevalence.

Thus, there is a need in the field for alternative adenoviral vectors that are producible in large quantities, that do not encounter pre-existing immunities in the host, but that are still immunogenic and capable of inducing a strong immune response against the antigens encoded by the heterologous nucleic acids inserted in the vector.

### BRIEF SUMMARY OF THE INVENTION

Provided herein is an isolated nucleic acid sequence encoding a chimeric adenoviral capsid or a functional derivative thereof. The chimeric adenoviral capsid or functional derivative thereof can, for example, comprise a fiber polypeptide having an amino acid sequence with at least 95% identity to the amino acid sequence of SEQ ID NO:11, a hexon polypeptide having an amino acid sequence with at least 95% identity to the amino acid sequence of SEQ ID NO:12, and a penton polypeptide having an amino acid sequence with at least 95% identity to the amino acid sequence of SEQ ID NO:13. In certain embodiments, the fiber polypeptide sequence comprises the amino acid sequence of SEQ ID NO:11. In certain embodiments, the hexon polypeptide sequence comprises the amino acid sequence of SEQ ID NO:12. In certain embodiments, the penton polypeptide comprises the amino acid sequence of SEQ ID NO:13.

Also provided herein are vectors comprising the isolated nucleic acids described herein. In certain embodiments, the vector is an adenoviral vector.

In certain embodiments, the adenoviral vector further comprises an E1 deletion. In certain embodiments, the adenoviral vector further comprises an E3 deletion. In certain embodiments, the adenoviral vector is a chimeric adenoviral vector comprising one or more adenoviral nucleic acid sequences from at least one of human adenovirus-4, human adenovirus-5, human adenovirus-26, or human adenovirus-35. The adenoviral vector can, for example, comprise a human adenovirus-5 (HAdV-5) E4 orf6. In certain embodiments, the adenoviral vector can, for example, comprise a nucleic acid sequence selected from the group consisting of SEQ ID NO:8, SEQ ID NO:9, and SEQ ID NO:10.

In certain embodiments, the adenoviral vector further comprises at least one transgene. In certain embodiments, the at least one transgene is located at the E1 deletion, at the E3 deletion, and/or adjacent to the right inverted terminal repeat (rITR).

Also provided are recombinant cells comprising the adenoviral vectors described herein. Also provided are methods of producing the adenoviral vectors. The methods comprise (a) growing the recombinant cells described herein under conditions for production of the adenoviral vector; and (b) isolating the adenoviral vector from the recombinant cell.

Also provided are pharmaceutical compositions comprising the adenoviral vectors described herein and a pharmaceutically acceptable carrier.

Also provided are methods of inducing an immune response in a subject in need thereof. The methods comprise administering to the subject the pharmaceutical compositions described herein.

Also provided are methods of producing the pharmaceutical compositions, the methods comprise combining the adenoviral vectors described herein with a pharmaceutically acceptable carrier.

Also provided are methods of expressing a transgene in a subject in need thereof. The methods comprise (a) identifying a subject in need of the expressed transgene; (b) contacting the subject with an adenoviral vector comprising a transgene described herein; and (c) expressing the transgene in the subject. In certain embodiments, expression of the transgene in the subject in need thereof treats or prevents a disease or disorder. In certain embodiments, contacting the subject with the vector can, for example, comprise isolating a cell from the subject and contacting the cell with the vector. The subject can, for example, be a human subject.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows the full genome map of the Ad20-42-42 wild type virus (SEQ ID NO:1).

FIG. 2 shows a scheme for the construction of the adenovirus vector genome.

FIG. 3 shows a schematic of the multicloning site (MCS) in the adaptor plasmid.

FIG. 4 shows a schematic of the adaptor plasmid: pAdApt20-42-42.Empty.

FIG. 5 shows a schematic of the intermediate plasmid: pBR.Ad20-42-42.SbfI final interm.

FIG. 6 shows a schematic of the right-end plasmid: pBrAd20-42-42 SrfI-rITR.dE3.5orf6.

FIGS. 7A-7B show the cellular immune responses induced by Ad20-42-42.FLuc. FIG. 7A shows a schematic of the immunization procedure. FIG. 7B shows a graph demonstrating the cellular immune responses induced by Ad20-42-42.FLuc.

FIG. 8 shows that no major cross-neutralization was observed between Ad20-42-42 and Ad26 vectors.

FIG. 9 shows a graph demonstrating the results of seroprevalence of different adenoviral constructs in human subjects.

FIG. 10 shows a graph demonstrating transduction potential for Ad20-42-42, HAd5, and HAd35 vectors. Luciferase-expression is presented as relative light units (RLU) per milligram (mg) protein.

FIG. 11 shows representative LacZ staining for cells transduced with Ad5LacZ and Ad20-42-42LacZ. The transduction was performed with three vector doses: 10,000; 5,000, and 1,000 virus particles (VP) per cell with FX added.

FIGS. 12A-12B show graphs demonstrating HAdV5 and Ad20-42-42 transduction of CHO cells expressing or lacking CAR or Sialic acid, and TC1 cells expressing or lacking desmoglein 2 (DSG2).

FIGS. 12C-12D show graphs demonstrating HAdV5 and Ad20-42-42 transduction of CHO cells lacking (negative) or expressing different isoforms (BC1, BC2, C1, C2) of CD46.

FIG. 13 shows in vivo distribution of luciferase-encoding HAdV5 and Ad20-42-42 in mice. Mice were pre-treated without (CL−; top and bottom right panel) or with (CL+; top and bottom left panel) clodronate-treatment and injected intravenously with each vector. Control animals were injected with phosphate buffered saline (PBS). Luciferase expression goes from low to high.

FIGS. 14A-D shows the quantification of the AdV vectors (HAdV-5 and Ad20-42-42 respectively). DNA copy numbers after intravenous transduction of mice are shown, in the absence (CL−; FIGS. 14A and 14C) or presence (CL+; FIGS. 14B and 14D) of clodronate. Vector genomes were quantified by qPCR.

### DETAILED DESCRIPTION OF THE INVENTION

This disclosure is based upon, at least in part, the isolation of chimeric adenoviral vectors comprising a chimeric capsid polypeptide or a functional derivative thereof, wherein the chimeric capsid polypeptide comprises a fiber and hexon polypeptide from a first adenovirus (e.g., human adenovirus-42) and a penton polypeptide from a second adenovirus (e.g., human adenovirus-20). The adenoviral vectors are capable of eliciting an immune response, while maintaining low seroprevalence. The adenoviral vectors can be formulated for vaccines and used to induce protective immunity against specific antigens of interest. The adenoviral vectors can also be constructed to express a transgene of interest in a subject in need thereof.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., hexon and fiber polypeptides and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “protective immunity” or “protective immune response” means that the vaccinated subject is able to control an infection with the pathogenic agent against which the vaccination was done. The pathogenic agent can, for example, be an antigenic gene product or antigenic protein, or a fragment thereof. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

The term “adjuvant” is defined as one or more substances that cause stimulation of the immune system. In this context, an adjuvant is used to enhance an immune response to the adenovirus vectors of the invention.

As used herein, the term “antigenic gene product or fragment thereof” or “antigenic protein” can include a bacterial, viral, parasitic, or fungal protein, or a fragment thereof. Preferably, an antigenic protein or antigenic gene product is capable of raising a protective immune response in a host, e.g., inducing an immune response against a disease or infection (e.g., a bacterial, viral, parasitic, or fungal disease or infection), and/or producing an immunity in (i.e., vaccinating) a subject against a disease or infection, that protects the subject against the disease or infection.

As used herein, the term “chimeric” means a gene, nucleic acid, protein, peptide or polypeptide that comprises two or more genes, nucleic acids, proteins, peptides or polypeptides not normally associated together. A “chimeric” gene, nucleic acid, or protein can be a fusion between two or more unrelated sequences (e.g., two or more distinct nucleic acids that encode two or more distinct proteins). A “chimeric” gene, nucleic acid, or protein can be a fusion between two or more related sequences (e.g., the nucleic acids encode the same protein, however, the nucleic acids are derived from a different source material, i.e., one nucleic acid is from one human adenovirus and the other nucleic acid is from a second unrelated human adenovirus).

### Adenoviral Vectors

Exposure to certain adenoviruses has resulted in immune responses against certain adenoviral serotypes, which can affect efficacy of adenoviral vectors. Because infections with human adenoviruses are common in humans, the prevalence of neutralizing antibodies against human adenoviruses in human populations is high. The presence of such neutralizing antibodies in individuals may be expected to reduce the efficacy of a gene transfer vector based on a human adenoviral backbone. One way to circumvent the reduction of efficacy is to replace the epitopes on the adenoviral capsid proteins that are the targets of neutralizing antibodies. The target sequences on the capsid proteins can be replaced with protein sequences from other adenoviruses (e.g., chimeric adenoviruses of multiple human adenoviruses) which are of low prevalence, and therefore against which neutralizing antibodies are rare in human populations.

A “capsid protein” refers to a protein on the capsid of an adenovirus (e.g., AD20 and/or AD42) or a functional fragment or derivative thereof that is involved in determining the serotype and/or tropism of an adenovirus. Capsid proteins typically include the fiber, penton, and/or hexon proteins. In certain embodiments, the capsid protein is an entire or full-length capsid protein of the adenovirus. In other embodiments, the capsid protein is a fragment or a derivative of a full-length capsid protein of the adenovirus. In certain embodiments, the hexon, penton and fiber encoded by an adenoviral vector of the invention are from a different adenoviral background.

A “chimeric adenoviral capsid” as used herein, refers to a capsid of adenoviral origin, which comprises a fiber, a penton, and/or a hexon polypeptide, wherein the fiber, penton, and/or hexon polypeptide are derived from different adenoviral origins (e.g., an Ad42 fiber and hexon polypeptide and an Ad20 penton polypeptide).

A “hexon polypeptide” refers to adenovirus hexon coat proteins, functional fragments, and derivatives thereof.

A “fiber polypeptide” refers to adenovirus fiber proteins, functional fragments, and derivatives thereof.

A “penton polypeptide” refers to adenovirus penton proteins, functional fragments, and derivatives thereof.

One target of neutralizing antibodies against adenoviruses is the major coat protein, the hexon protein. Replacing the hexon protein or variable sequences within the hexon protein, which define serotype and bind to neutralizing antibodies, with the hexon protein or variable sequences within the hexon protein from adenoviruses that are rare in the human population can allow for the construction of adenovirus vectors that would be less susceptible to neutralization by antibodies commonly found in humans.

Hexon hypervariable regions (HVRs) are regions of the hexon polypeptide representing the highest variability among the different adenoviral serotypes. In general, these HVRs are thought to correspond to the solvent-exposed surfaces of the hexon protein trimer (within the context of the intact viral particle) and, relatedly, they are expected to be important determinants of antibody-mediated adenovirus neutralization (Roberts et al., Nature 441:239-43 (2006)). Replacement of the hexon HVRs of a given adenoviral vector by those of an adenovirus with low (or no) seroprevalence in humans therefore represents a possible means to circumvent pre-existing anti-vector humoral immunity in human target populations. Consequently, there have been multiple studies exploring the concept of hexon-chimerism, mostly involving hexon sequence replacements within HAdV-5-based vectors (Roy et al., J Virol. 72:6875-9 (1998); Gall et al., J Virol. 72:10260-4 (1998); Youil et al., Hum. Gene Ther. 13:311-20 (2002); Wu et al. J Virol. 76:12775-82 (2002); Roy et al., Virology. 333:207-14 (2005); Roberts et al., Nature 441:239-43 (2006); Bradley et al., J Virol. 86:1267-72 (2012); Yu et al., Biochem Biophys Res Commun. 421:170-6 (2012); Bruder et al, PLoS One. 7(4):e33920 (2012)).

A second target of neutralizing antibodies against adenoviruses is the fiber protein. Replacing the fiber protein with fiber sequences from rare adenoviruses of human origin, more preferably replacing the variable sequences within the fiber protein, can also allow for the construction of adenovirus vectors that would be less susceptible to neutralization by antibodies commonly found in humans. A combination of the fiber replacement with hexon replacements described above can confer additional resistance to neutralization by antibodies commonly present in human populations.

A third target of neutralizing antibodies against adenoviruses is the penton protein. Replacing the penton protein with penton sequences from rare adenoviruses of human origin can also allow for the construction of adenovirus vectors that would be less susceptible to neutralization by antibodies commonly found in humans. A combination of hexon replacements, fiber replacements, and penton replacements described above can confer additional resistance to neutralization by antibodies commonly present in human populations.

This disclosure provides isolated nucleic acid sequences encoding chimeric adenoviral capsids or functional derivatives thereof. The chimeric adenoviral capsid or functional derivative thereof can, for example, comprise a fiber polypeptide, a hexon polypeptide, and a penton polypeptide. The fiber and hexon polypeptide can, for example, be derived from a first adenovirus (e.g., human adenovirus-42) and the penton polypeptide can, for example, be derived from a second adenovirus (e.g., human-adenovirus-20).

A “functional derivative” of a polypeptide suitably refers to a modified version of a polypeptide, e.g. wherein one or more amino acids of the polypeptide may be deleted, inserted, modified and/or substituted. A derivative of an unmodified adenoviral capsid protein is considered functional if, for example (a) an adenovirus comprising the derivative capsid protein within its capsid retains substantially the same or a lower seroprevalence compared to an adenovirus comprising the unmodified capsid protein; and/or, (b) an adenovirus comprising the derivative capsid protein within its capsid retains substantially the same or a higher host cell infectivity compared to an adenovirus comprising the unmodified capsid protein; and/or (c) an adenovirus comprising the derivative capsid protein within its capsid retains substantially the same or a higher immunogenicity compared to an adenovirus comprising the unmodified capsid protein; and/or (d) an adenovirus comprising the derivative capsid protein within its capsid retains substantially the same or a higher level of transgene productivity compared to an adenovirus comprising the unmodified capsid protein.

An “adenoviral vector” refers to a recombinant vector derived from or containing at least a portion of an adenoviral genome.

In preferred embodiments, the chimeric adenoviral capsid can, for example, comprise a fiber polypeptide having an amino acid sequence with at least 90%, at least 91%, at least 92%, at least 93%, at least 94%, at least 95%, at least 96%, at least 97%, at least 98%, or at least 99% identity to the amino acid sequence of SEQ ID NO:11; a hexon polypeptide having an amino acid sequence with at least 90%, at least 91%, at least 92%, at least 93%, at least 94%, at least 95%, at least 96%, at least 97%, at least 98%, or at least 99% identity to the amino acid sequence of SEQ ID NO:12; and a penton polypeptide having an amino acid sequence with at least 90%, at least 91%, at least 92%, at least 93%, at least 94%, at least 95%, at least 96%, at least 97%, at least 98%, or at least 99% identity to the amino acid sequence of SEQ ID NO:13. In certain embodiments, the fiber polypeptide sequence comprises the amino acid sequence of SEQ ID NO:11. In certain embodiments, the hexon polypeptide sequence comprises the amino acid sequence of SEQ ID NO:12. In certain embodiments, the penton polypeptide comprises the amino acid sequence of SEQ ID NO:13

In preferred embodiments, provided are vectors, preferably adenoviral vectors, comprising the isolated nucleic acids disclosed herein. The adenoviral vectors comprise the isolated nucleic acids encoding a chimeric adenoviral capsid or functional derivative thereof, wherein the chimeric adenoviral capsid or functional derivative thereof comprises a fiber polypeptide having an amino acid sequence with at least 95% identity to the amino acid sequence of SEQ ID NO:11, a hexon polypeptide having an amino acid sequence with at least 95% identity to the amino acid sequence of SEQ ID NO:12, and a penton polypeptide having an amino acid sequence with at least 95% identity to the amino acid sequence of SEQ ID NO:13.

Typically, an adenoviral vector of the invention comprises the entire recombinant adenoviral genome on, e.g., a plasmid, cosmid, or baculovirus vector. The nucleic acid molecules of the invention can be in the form of RNA or in the form of DNA obtained by cloning or produced synthetically. The DNA can be double-stranded or single-stranded.

One of ordinary skill will recognize that elements derived from multiple serotypes can be combined in a single adenoviral vector, for example human or simian adenovirus. Thus, a chimeric adenovirus vector that combines desirable properties from different serotypes can be produced. Thus, in some embodiments, a chimeric adenovirus vector of the invention could combine the absence of pre-existing immunity of a chimeric hexon and/or fiber polypeptide sequences with the high-level antigen and/or transgene delivery and presentation capacity of an existing adenoviral vector, such as rAd4, rAd5, rAd26, or rAd35.

Advantages of adenoviral vectors for use as vaccines and/or as a vehicle for transgene expression can include, but is not limited to, ease of manipulation, good manufacturability at large scale, and an excellent safety record based on many years of experience in research, development, manufacturing and clinical trials with numerous adenoviral vectors that have been reported. Adenoviral vectors that are used as vaccines generally provide a good immune response to the transgene-encoded protein or transgene encoded antigenic gene product, including a cellular immune response. An adenoviral vector according to the invention can be based on any type of adenovirus, and in certain embodiments is a human adenovirus, which can be of any group or serotype. In preferred embodiments, the recombinant adenovirus is based upon a human adenovirus from group A, B, C, D, E, F, or G. In other preferred embodiments, the recombinant adenovirus is based upon a human adenovirus serotype 5, 11, 26, 34, 35, 48, 49, or 50. In other embodiments, it is a simian adenovirus, such as chimpanzee or gorilla adenovirus, which can be of any serotype. In certain embodiments, the recombinant adenovirus is based upon chimpanzee adenovirus type 1, 3, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50, 67, or SA7P.

In a more preferred embodiment, the chimpanzee adenovirus vector of the second composition is ChAdV3. Recombinant chimpanzee adenovirus serotype 3 (ChAd3 or cAd3) is a subgroup C adenovirus with properties similar to those of human adenovirus serotype 5 (Ad5). ChAd3 has been shown to be safe and immunogenic in human studies evaluating candidate vaccines for hepatitis C virus (HCV) (Barnes E, et al. 2012 Science translational medicine 4: 115ra1). It was reported that ChAd3-based vaccines were capable of inducing an immune response comparable to a human Ad5 vectored vaccine. See, e.g., Peruzzi D, et al. 2009 Vaccine 27: 1293-300 and Quinn K M, et al. 2013 J Immunol 190: 2720-35; WO 2005/071093; WO2011/0130627, etc.

Adenoviral vectors, methods for construction thereof and methods for propagating thereof, are well known in the art and are described in, for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication,” M. S. Horwitz, “Adenoviruses,” Chapters 67 and 68, respectively, in Virology, B. N. Fields et al., eds., 3d ed., Raven Press, Ltd., New York (1996), and other references mentioned herein. Typically, construction of adenoviral vectors involves the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DNA, 2d ed., Scientific American Books (1992), and Ausubel et al., Current Protocols in Molecular Biology, Wiley Interscience Publishers, NY (1995), and other references mentioned herein.

In certain embodiments, the adenoviral vector comprises an E1 deletion and/or an E3 deletion. An E1 or E3 deletion can, for example, include a complete deletion of the gene or a partial deletion, which renders the E1 or E3 gene product functionally defective. Thus, in certain embodiments, the adenovirus is replication deficient, e.g., because it contains a deletion in the E1 region of the genome. As known to the skilled person, in case of deletions of essential regions from the adenovirus genome, the functions encoded by these regions have to be provided in trans, preferably by the producer cell, i.e., when parts or whole of E1, E2 and/or E4 regions are deleted from the adenovirus, these have to be present in the producer cell, for instance integrated in the genome thereof, or in the form of so-called helper adenovirus or helper plasmids. The adenovirus may also have a deletion in the E3 region, which is dispensable for replication, and hence such a deletion does not have to be complemented. One or more of the E1, E2, E3, and E4 regions can also be inactivated by other means, such as by inserting a transgene of interest (usually linked to a promoter) into the regions to be inactivated.

A producer cell (sometimes also referred to in the art and herein as ‘packaging cell’ or ‘complementing cell’) that can be used can be any producer cell wherein a desired adenovirus can be propagated. For example, the propagation of recombinant adenovirus vectors is done in producer cells that complement deficiencies in the adenovirus. Such producer cells preferably have in their genome at least an adenovirus E1 sequence, and thereby are capable of complementing recombinant adenoviruses with a deletion in the E1 region. Any E1-complementing producer cell can be used, such as human retina cells immortalized by E1, e.g. 911 or PER.C6 cells (see U.S. Pat. No. 5,994,128), E1-transformed amniocytes (See EP patent 1230354), E1-transformed A549 cells (see e.g. WO 98/39411, U.S. Pat. No. 5,891,690), GH329:HeLa (Gao et al., 2000, Hum Gene Ther 11: 213-19), 293, and the like. In certain embodiments, the producer cells are for instance HEK293 cells, or PER.C6 cells, or 911 cells, or IT293SF cells, and the like. Production of adenoviral vectors in producer cells is reviewed in (Kovesdi et al., 2010, Viruses 2: 1681-703).

In certain embodiments, the adenoviral vector is a chimeric adenoviral vector comprising one or more human adenoviral nucleic acid sequences. The human adenoviral nucleic acids can, for example, be selected from human adenovirus-4 (Ad-4), human adenovirus-5 (Ad-5), human adenovirus-26 (Ad-26), or human adenovirus-35 (Ad-35). In certain embodiments, an E1-deficient adenoviral vector comprises the E4-orf6 coding sequence of an adenovirus of human Ad5. This allows propagation of such adenoviruses in well known complementing cell lines that express the E1 genes of Ad5, such as, for example, 293 cells or PER.C6 cells (see, e.g. Fallaux et al., 1998, Hum Gene Ther 9: 1909-17, Havenga et al., 2006, J Gen Virol 87: 2135-43; WO 03/104467, incorporated in their entirety by reference herein).

In certain embodiments, the adenoviral vector comprises a transgene. A “transgene” refers to a heterologous nucleic acid, which is a nucleic acid that is not naturally present in the vector, and according to the present invention the transgene can encode an antigenic gene product or antigenic protein that elicits an immune response in the subject. The transgene can also encode a therapeutic protein to treat or prevent a disease in a subject in need thereof. The transgene can, for example, be introduced into the vector by standard molecular biology techniques. The transgene can, for example, be cloned into a deleted E1 or E3 region of an adenoviral vector, or in the region between the E4 region and the rITR. A transgene is generally operably linked to expression control sequences. In preferred embodiments, the transgene is inserted at a transgene insertion site.

If required, the chimeric adenoviral capsid sequence comprising the fiber, hexon, and penton polypeptide sequences according to embodiments of the invention, and/or the transgene can be codon-optimized to ensure proper expression in the treated host (e.g., human). Codon-optimization is a technology widely applied in the art.

The transgene can be under the control of (i.e., operably linked to) an adenovirus-derived promoter (e.g., the Major Late Promoter) or can be under the control of a heterologous promoter. Examples of suitable heterologous promoters include the CMV promoter and the RSV promoter. Preferably, the promoter is located upstream of the heterologous gene of interest within an expression cassette.

In preferred embodiments, the adenoviral vector comprises a nucleic acid sequence selected from the group consisting of SEQ ID NO:8, SEQ ID NO:9, and SEQ ID NO:10.

### Pharmaceutical and/or Immunogenic Compositions

In another general aspect, provided are pharmaceutical compositions comprising an isolated polynucleotide of the invention, an isolated polypeptide of the invention, a vector of the invention, an adenoviral vector of the invention, and/or a host cell of the invention and a pharmaceutically acceptable carrier. The term “pharmaceutical composition” as used herein means a product comprising an isolated polynucleotide of the invention, an isolated polypeptide of the invention, an isolated vector (e.g., adenoviral vector) of the invention, and/or a host cell of the invention together with a pharmaceutically acceptable carrier. Polynucleotides, polypeptides, vectors, and/or host cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, vector, and/or host cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

Pharmaceutical compositions can, for example, be formulated for the expression of a transgene in a subject in need thereof (i.e., a pharmaceutical composition designed for transgene expression in a subject in need thereof). Pharmaceutical compositions can, for example, be formulated for the expression of an antigen polypeptide or an antigenic fragment thereof (e.g., a pharmaceutical composition to elicit an immune response in a subject in need thereof).

Pharmaceutical composition designed to elicit an immune response in a subject in need thereof can, for example, be referred to as an immunogenic composition. Immunogenic compositions are compositions comprising an immunologically effective amount of purified or partially purified adenoviral vectors for use in the invention. Said compositions can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

The immunogenic compositions according to embodiments of the present invention can be made using methods known to those of skill in the art in view of the present disclosure. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol, or polyethylene glycol can be included.

The immunogenic compositions useful in the invention can comprise adjuvants. Adjuvants suitable for co-administration in accordance with the invention should be ones that are potentially safe, well tolerated, and effective in subjects including QS-21, Detox-PC, MPL-SE, MoGM-CSF, TiterMax-G, CRL-1005, GERBU, TERamide, PSC97B, Adjumer, PG-026, GSK-I, AS01, AS03, AS04, AS15, GcMAF, B-alethine, MPC-026, Adjuvax, CpG ODN, Betafectin, Alum, and MF59.

Other adjuvants that can be administered include lectins, growth factors, cytokines and lymphokines such as alpha-interferon, gamma interferon, platelet derived growth factor (PDGF), granulocyte-colony stimulating factor (gCSF), granulocyte macrophage colony stimulating factor (gMCSF), tumor necrosis factor (TNF), epidermal growth factor (EGF), IL-I, IL-2, IL-4, IL-6, IL-8, IL-10, and IL-12 or encoding nucleic acids therefore.

The compositions of the invention can comprise a pharmaceutically acceptable excipient, carrier, buffer, stabilizer, or other materials well known to those skilled in the art. Such materials should be non-toxic and should not interfere with the efficacy of the active ingredient. The precise nature of the carrier or other material can depend on the route of administration, e.g., intramuscular, subcutaneous, oral, intravenous, cutaneous, intramucosal (e.g., gut), intranasal or intraperitoneal routes.

### Method for Inducing Protective Immunity and/or Expressing a Transgene

Another general aspect of the invention relates to a method of inducing an immune response and/or expressing a transgene in a subject in need thereof. The methods can, for example, comprise identifying a subject in need thereof; contacting the subject in need thereof with a pharmaceutical and/or immunogenic composition described herein; and eliciting an immune response and/or expressing a transgene in the subject in need thereof. In certain embodiments, the methods can, for example, comprise administering to the subject a vaccine comprising an adenoviral vector described herein and a pharmaceutically acceptable carrier.

Also provided herein are methods of producing a vaccine. The methods comprise combining an adenoviral vector described herein with a pharmaceutically acceptable carrier.

Any of the immunogenic compositions according to embodiments of the invention, including but not limited to those described herein, can be used in methods of the invention as a vaccine. Any of the pharmaceutical compositions accordingly to embodiments of the invention, including, but not limited to those described herein, can be used in methods of the invention to treat or prevent a disease in a subject in need thereof by expressing a transgene of interest.

Administration of the immunogenic compositions/vaccines/pharmaceutical compositions comprising the vectors is typically intramuscular or subcutaneous. However other modes of administration such as intravenous, cutaneous, intradermal or nasal can be envisaged as well. Intramuscular administration of the immunogenic compositions can be achieved by using a needle to inject a suspension of the adenovirus vector. An alternative is the use of a needleless injection device to administer the composition (using, e.g., BIOJECTOR®) or a freeze-dried powder containing the vaccine.

For intravenous, cutaneous or subcutaneous injection, or injection at the site of affliction, the vector will be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

Typically, administration will have a prophylactic aim to generate an immune response against an antigen of interest (e.g., a bacterial, viral, parasitic, and/or fungal pathogen) before infection or development of symptoms. Administration of an adenoviral vector expressing a transgene of interest can also have a prophylactic aim in a subject in need thereof. For example, a subject in need thereof could have reduced or eliminated endogenous expression of the gene corresponding to the transgene of interest. Diseases and disorders that can be treated or prevented in accordance with the invention include those in which an immune response can play a protective or therapeutic role and/or corrective expression of the transgene results in the normal functioning of the cells in the subject in need thereof. In other embodiments, the adenovirus vectors can be administered for post-exposure prophylactics.

The immunogenic compositions containing the chimeric human adenovirus vectors are administered to a subject, giving rise to an immune response to the antigen of interest in the subject. An amount of a composition sufficient to induce a detectable immune response is defined to be an “immunologically effective dose” or an “effective amount” of the composition. The immunogenic compositions of the invention can induce a humoral as well as a cell-mediated immune response. In a typical embodiment the immune response is a protective immune response.

The pharmaceutical compositions can be administered to a subject in need thereof in a therapeutically effective amount to treat or prevent a disease. A therapeutically effective amount means an amount of the adenoviral vector expressing the transgene of interest that results in the treatment of a disease, disorder, or condition; an amount that prevents or slows the progression of the disease, disorder, or condition; or an amount that reduces or completely alleviates symptoms associated with the disease, disorder, or condition.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

As used herein, the terms “treat,” “treating,” and “treatment” are all intended to refer to an amelioration or reversal of at least one measurable physical parameter related to the disease, disorder or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. ed., 1980.

Following production of adenovirus vectors and optional formulation of such particles into compositions, the vectors can be administered to an individual, particularly a human or another primate. Administration can be to humans, or another mammal, e.g., mouse, rat, hamster, guinea pig, rabbit, sheep, goat, pig, horse, cow, donkey, monkey, dog, or cat. Delivery to a non-human mammal need not be for a therapeutic purpose, but can be for use in an experimental context, for instance in investigation of mechanisms of immune responses to the adenovirus vectors.

In one exemplary regimen, the adenoviral vector is administered (e.g., intramuscularly) in a volume ranging between about 100 μl to about 10 ml containing concentrations of about 10⁴ to 10¹² virus particles/ml. Preferably, the adenoviral vector is administered in a volume ranging between 0.1 and 2.0 ml. For example, the adenoviral vector can be administered with 100 μl, 500 μl, 1 ml, 2 ml. More preferably the adenoviral vector is administered in a volume of 0.5 ml. Optionally, the adenoviral vector can be administered in a concentration of about 10⁷ vp/ml, 10⁸ vp/ml, 10⁹ vp/ml, 10¹⁰ vp/ml, 5×10¹⁰ vp/ml, 10¹¹ vp/ml, or 10¹² vp/ml. Typically, the adenoviral vector is administered in an amount of about 10⁹ to about 10¹² viral particles (vp) to a human subject during one administration, more typically in an amount of about 10¹⁰ to about 10¹² vp. The initial administration can, for example, be followed by a boost as described above.

The initial administration can be followed by a boost or a kick from a vaccine/composition comprising the same adenoviral vector encoding an antigen of interest and/or transgene of interest or a vaccine/composition comprising a different adenoviral vector encoding the same antigen of interest and/or transgene of interest.

The composition can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration.

The compositions of the invention can be administered alone or in combination with other treatments, either simultaneously or sequentially dependent upon the condition to be treated.

### EMBODIMENTS

The invention provides also the following non-limiting embodiments.

Embodiment 1 is an isolated nucleic acid sequence encoding a chimeric adenoviral capsid or a functional derivative thereof, wherein the chimeric adenoviral capsid or functional derivative thereof comprises a fiber polypeptide having an amino acid sequence with at least 95% identity to the amino acid sequence of SEQ ID NO:11, a hexon polypeptide having an amino acid sequence with at least 95% identity to the amino acid sequence of SEQ ID NO:12, and a penton polypeptide having an amino acid sequence with at least 95% identity to the amino acid sequence of SEQ ID NO:13.

Embodiment 2 is the isolated nucleic acid sequence of embodiment 1, wherein the fiber polypeptide sequence comprises the amino acid sequence of SEQ ID NO:11.

Embodiment 3 is the isolated nucleic acid of embodiment 1 or 2, wherein the hexon polypeptide sequence comprises the amino acid sequence of SEQ ID NO:12.

Embodiment 4 is the isolated nucleic acid of any one of embodiments 1 to 3, wherein the penton polypeptide comprises the amino acid sequence of SEQ ID NO:13.

Embodiment 5 is a vector comprising the isolated nucleic acid of any one of embodiments 1 to 4.

Embodiment 6 is the vector of embodiment 5, wherein the vector is an adenoviral vector.

Embodiment 7 is the vector of embodiment 6, wherein the adenoviral vector further comprises a transgene, optionally wherein the transgene is a therapeutic transgene.

Embodiment 8 is the vector of embodiment 6 or 7, wherein the adenoviral vector further comprises an E1 deletion.

Embodiment 9 is the vector of any one of embodiments 6 to 8, wherein the adenoviral vector further comprises an E3 deletion.

Embodiment 10 is the vector of any one of embodiments 6 to 9, wherein the adenoviral vector is a chimeric adenoviral vector comprising one or more adenoviral nucleic acid sequences from at least one of human adenovirus-4, human adenovirus-5, human adenovirus-26, or human adenovirus-35.

Embodiment 11 is the vector of embodiment 10, wherein the adenoviral vector comprises a human adenovirus-5 (HAdV-5) E4 orf6.

Embodiment 12 is the vector of any one of embodiments 6 to 10, wherein the adenoviral vector comprises a nucleic acid sequence selected from the group of SEQ ID NO:8, SEQ ID NO:9, and SEQ ID NO:10.

Embodiment 13 is the vector of any one of embodiments 6 to 12, wherein the transgene is located at the E1 deletion, at the E3 deletion, and/or adjacent to the right inverted terminal repeat (rITR).

Embodiment 14 is a recombinant cell comprising the vector of any one of embodiments 5 to 12.

Embodiment 15 is a method of producing a vector, comprising:
(a) growing the recombinant cell of embodiment 14 under conditions for production of the adenoviral vector; and
(b) isolating the vector from the recombinant cell.

Embodiment 16 is an immunogenic composition comprising the adenoviral vector of any one of embodiments 6 to 13 and a pharmaceutically acceptable carrier.

Embodiment 17 is a method of inducing an immune response in a subject in need thereof, the method comprising administering to the subject the immunogenic composition of embodiment 16.

Embodiment 18 is a method of producing a vaccine, the method comprising combining an adenoviral vector of any one of embodiments 6 to 13 with a pharmaceutically acceptable carrier.

Embodiment 19 is an adenoviral vector comprising (a) at least one transgene; and (b) a nucleic acid encoding a chimeric adenoviral capsid or a functional derivative thereof, wherein the chimeric adenoviral capsid comprises a fiber polypeptide having an amino acid sequence with at least 95% identity to the amino acid sequence of SEQ ID NO:11, a hexon polypeptide having an amino acid sequence with at least 95% identity to the amino acid sequence of SEQ ID NO:12, and a penton polypeptide having an amino acid sequence with at least 95% identity to the amino acid sequence of SEQ ID NO:13.

Embodiment 20 is the adenoviral vector of embodiment 19, wherein the fiber polypeptide sequence comprises the amino acid sequence of SEQ ID NO:11.

Embodiment 21 is the adenoviral vector of embodiment 19 or 20, wherein the hexon polypeptide sequence comprises the amino acid sequence of SEQ ID NO:12.

Embodiment 22 is the adenoviral vector of any one of embodiments 19 to 21, wherein the penton polypeptide comprises the amino acid sequence of SEQ ID NO:13.

Embodiment 23 is the adenoviral vector of any one of embodiments 19 to 22, wherein the adenoviral vector further comprises an E1 deletion.

Embodiment 24 is the adenoviral vector of any one of embodiments 19 to 23, wherein the adenoviral vector further comprises an E3 deletion.

Embodiment 25 is the adenoviral vector of any one of embodiments 19 to 24, wherein the adenoviral vector is a chimeric adenoviral vector comprising one or more adenoviral nucleic acid sequences from at least one of human adenovirus-4, human adenovirus-5, human adenovirus-26, or human adenovirus-35.

Embodiment 26 is the adenoviral vector of embodiment 25, wherein the adenoviral vector comprises a human adenovirus-5 (HAdV-5) E4 orf6.

Embodiment 27 is the adenoviral vector of any one of embodiments 19 to 26, wherein the adenoviral vector comprises a nucleic acid sequence selected from the group of SEQ ID NO:8, SEQ ID NO:9, and SEQ ID NO:10.

Embodiment 28 is the adenoviral vector of any one of embodiments 19 to 27, wherein the transgene is located at the E1 deletion, at the E3 deletion, and/or adjacent to the right inverted terminal repeat (rITR).

Embodiment 29 is the adenoviral vector of any one of embodiments 19 to 28, wherein the transgene is a therapeutic transgene.

Embodiment 30 is a recombinant cell comprising the adenoviral vector of any one of embodiments 19 to 29.

Embodiment 31 is a method of producing an adenoviral vector, comprising:
(a) growing the recombinant cell of embodiment 30 under conditions for production of the adenoviral vector; and
(b) isolating the adenoviral vector from the recombinant cell.

Embodiment 32 is a pharmaceutical composition comprising the adenoviral vector of any one of embodiments 19 to 29 and a pharmaceutically acceptable carrier.

Embodiment 33 is a method of inducing an immune response in a subject in need thereof, the method comprising administering to the subject the pharmaceutical composition of embodiment 32.

Embodiment 34 is a method of producing a vaccine, the method comprising combining an adenoviral vector of any one of embodiments 19 to 29 with a pharmaceutically acceptable carrier.

Embodiment 35 is a method of expressing a transgene in a subject in need thereof, the method comprising:
a. identifying a subject in need of the expressed transgene;
b. contacting the subject with the vector of embodiment 7 or adenoviral vector of any one of embodiments 19 to 29; and
c. expressing the transgene in the subject.

Embodiment 36 is the method of embodiment 35, wherein expression of the transgene in the subject in need thereof treats or prevents a disease or disorder.

Embodiment 37 is the method of embodiment 35, wherein contacting the subject with the vector comprises isolating a cell from the subject and contacting the cell with the vector.

Embodiment 38 is the method of any one of embodiments 35 to 37, wherein the subject in need thereof is a human subject.

Embodiment 39 is the use of the vector of embodiment 7 to treat or prevent a disease or disorder in a subject in need thereof, the method comprising contacting the subject in need thereof with the vector, wherein contacting the subject in need thereof results in the expression of the therapeutic transgene.

Embodiment 40 is the use according to embodiment 39, wherein contacting the subject with the vector comprises isolating a cell from the subject and contacting the cell with the vector.

Embodiment 41 is the use of the adenoviral vector of any one of embodiments 19 to 29 to treat or prevent a disease or disorder in a subject in need thereof, the method comprising contacting the subject in need thereof with the adenoviral vector, wherein contacting the subject in need thereof results in the expression of the therapeutic transgene.

Embodiment 42 is the use of embodiment 41, wherein contacting the subject with the adenoviral vector comprises isolating a cell from the subject and contacting the cell with the adenoviral vector.

### EXAMPLES

### Example 1

### Generation of E1- and E3 Deleted Vectors Based on Novel Adenovirus Isolate Ad20-42-42

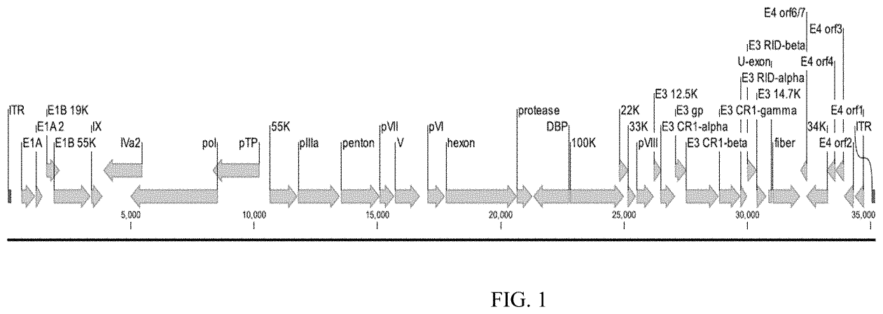

A novel human adenovirus isolate, Ad20-42-42 (SEQ ID NO:1), was identified and sequenced. This human adenovirus isolate was found to phylogenetically belong to the human adenovirus species D (HAdV-D) and it is a natural chimera of HAdV-20 and HAdV-42. The penton gene is originated from HAdV-20, while the hexon and the fiber genes are from HAdV-42. Shown in FIG. 1 is the genome map of the Ad20-42-42 human adenovirus isolate.

### Description of the Three-Plasmid-System Used for the Generation of Ad20-42-42-based Ad Vectors

The Ad20-42-42-based recombinant adenoviral vectors were generated by using a three-plasmid-system. The plasmid system consists of “adaptor plasmids” covering the 5′-end of the adenovirus genome in which the E1 region is deleted and replaced by an expression cassette carrying a gene of interest (LacZ, Luc+ or eGFP). The second, “intermediate” plasmid covers the middle part of the adenovirus genome without any modifications. The 3′-end of the adenovirus sequence is carried by the “right-end” plasmid. In the right-end construct the E3 region is deleted and the native ORF6/7 in the E4 region is replaced by the ORF6/7 of HAdV-5.

TABLE 1
Plasmids used to construct Ad20-42-42-based recombinant adenoviral vector

| | SEQ | | SEQ | | SEQ |
|---|---|---|---|---|---|
| Adaptor plasmid | ID NO: | Intermediate plasmid | ID NO: | Right end plasmid | ID NO: |
| pAdApt20-42-42.Empty | 2 | pBR.Ad20-42- | 6 | pBrAd20_42_42 SrfI- | 7 |
| pAdApt20-42-42.LacZ | 3 | 42.SbfI final interm | | rITR_dE3_5orf6 | |
| pAdApt20-42-42.Luc+ | 4 | | | | |
| pAdApt20-42-42.eGFP | 5 | | | | |

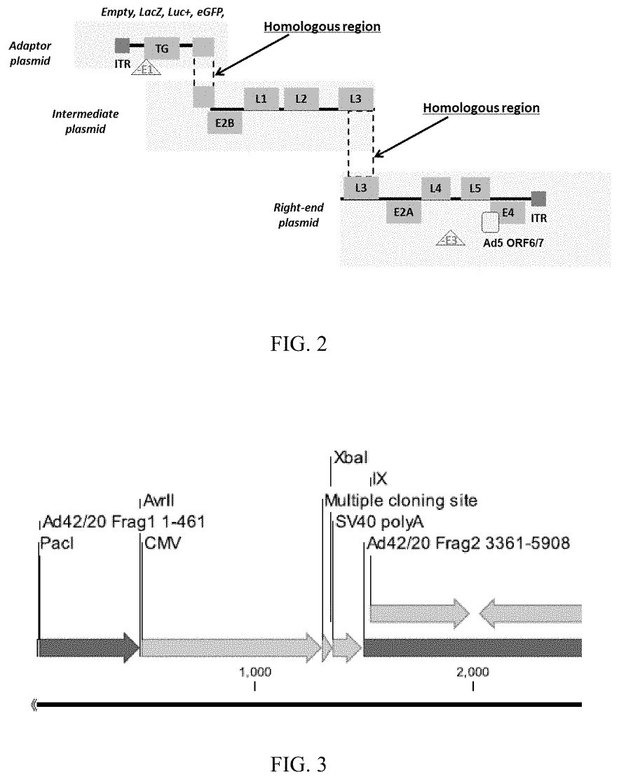

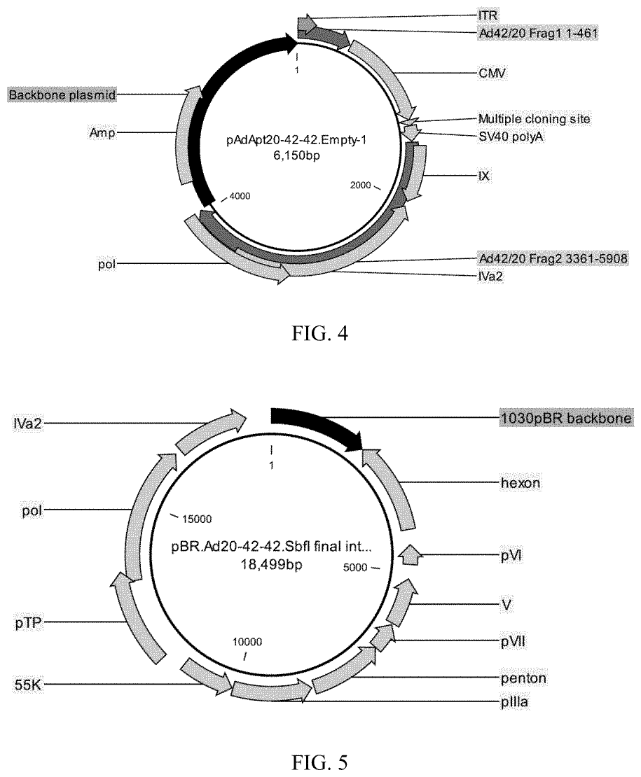

The plasmids carried virus vector sequences that were overlapping with each other on ˜2000 nucleotide positions to allow homologous recombination between these sequences in HEK293 or PER.C6® cells (FIG. 2).

Ad20-42-42-based Ad vector genome design: The plasmid systems were constructed by several steps of standard molecular cloning procedures. Ad20-42-42-based Ad vector genomes were each designed to comprise an E1 deletion, an E3 deletion, different transgene insertion sites, and a replacement of the native E4 open reading frame (orf) 6 and orf6/7 with that of human adenovirus-5 (HAdV-5) (base pairs 32966-34077 of GenBank sequence AC_000008).

### Adaptor Plasmids

First an E1 deleted Adaptor plasmid “pAdApt20-42-42.Empty” was constructed. An expression cassette was placed at the former location of the deleted E1 region. This cassette is driven by the cytomegalovirus major immediate early promoter (i.e. the “CMV promoter”) and contains an SV40-derived polyadenylation signal (“SV40 poly A”). The expression cassette is equipped by a multicloning site to facilitate the insertion of diverse genes of interest (LacZ, Luc+, and eGFP). The empty adaptor plasmid, pAdApt20-42-42 was constructed as follows.

Fragment 1 covering the wild type adenovirus sequence from nt 1-461 was generated with a 5′-flanking PacI restriction enzyme site and a 3′-flanking AvrII site introduced by PCR primers. The PCR product of Fragment 1 was double digested with PacI and AvrII.

Fragment 2, comprising SV40 polyA and nucleotides 3361-5908 of the wild type adenovirus sequence, was generated by preparing two PCR amplicons followed by the assembly PCR of these two products. The first PCR product containing an “SV40 polyA” was amplified from a previously constructed plasmid (pAdApt26.Empty; Abbink et al., J. Virol. 81(9):4654-63 (2007)). On the 5′-end of the PCR product an XbaI restriction site was introduced, while with the reverse primer an Ad20-42-42 homologue fragment was introduced in the PCR product. This overlap contained a natural XbaI recognition site (being present in the Ad20-42-42 genome) but it was purposely destroyed by changing one base at the recognition site). This PCR fragment was 174 bp long. The second PCR covers the Ad20-42-42 genome from the beginning of pIX (including pIX) to approximately the middle of the polymerase gene. The forward primer of this PCR contained an overlap with PCR product 1 containing the SV40 polyA (with the XbaI recognition destroyed). A PacI recognition site was introduced on this PCR fragment with the reverse primer. This PCR fragment was 2574 bp long. Afterwards an assembly PCR was performed using PCR product 1 and 2 as a template in order to merge these fragments. The product size of the assembly PCR was 2710 bp. Subsequently this assembly PCR product was double digested with XbaI and PacI.

The previously constructed plasmid (Abbink et al., J. Virol. 81(9):4654-63 (2007)) was digested with XbaI, AvrII and PacI to obtain the plasmid backbone and CMV promoter (fragment 2108 and 880). Afterwards a four-point ligation was performed using fragments of pAdApt26.Empty (fragments 2108 and 880) and Fragment 1 and Fragment 2. This resulted in pAdApt20-42-42.Empty plasmid.

Reporter genes were inserted in the MCS by either using the unique KpnI, HIndIII, or BamHI site together with the XbaI site followed by ligation.

### Intermediate Plasmid

The intermediate plasmid harbors the wild type adenovirus genome from nt position 2088 to 18494 without any modifications. First, two PCR fragments were created. One covering the 5′-end of the intermediate fragment with a PacI site incorporated in the forward primer, and the reverse primer was designed slightly downstream to a natural SbfI site (product size: 2273 bp) in the adenovirus genome. The second PCR fragment created with a forward primer designed slightly upstream from another natural SbfI site in the adenovirus genome, and a PacI site was incorporated in the reverse primer (product size: 2407 bp). Both PCR fragments were digested with PacI and SbfI restriction enzymes.

A pBR322 subclone backbone plasmid (Abbink et al., J. Virol. 81(9):4654-63 (2007)) with flanking PacI sites was digested with PacI (2086 bp). A 3-point ligation was performed using the two double digested PCR products and the PacI digested, dephosphorylated pBr backbone plasmid. This resulted in plasmid pBR.Ad20-42-42.PacI-SbfI. The pBR.Ad20-42-42.PacI-SbfI plasmid obtained in the previous step was cut open with SbfI restriction enzyme. The wild type Ad20-42-42 genomic DNA was digested with SbfI restriction enzyme and the 11858 nt long fragment covering the wild type genome from the polymerase gene up to approximately the middle of the pVI gene was ligated into the pBR. Ad20-42-42.PacI-SbfI plasmid. This resulted in “pBR.Ad20-42-42.SbfI final interm” plasmid.

### Right-end Plasmid

Construction of pBrAd20-42-42.SrfI-rITR: The 5′-end of the right-end of the Ad-20-42-42 genome was amplified by PCR (Fragment 1, 2098 bp) in a way that a natural SrfI-SbfI fragment was included in the PCR product and the forward primer was supplemented with a PacI recognition site. Another fragment covering the 3′-end of the right-end of the Ad-20-42-42 genome was generated with an SbfI recognition site introduced by the forward primer and with a PacI recognition site incorporated in the reverse primer. Close to the 5′-end of the PCR product a natural MluI site was present. Afterwards both PCR products were digested with PacI and SbfI enzymes.

A pBR322 subclone backbone plasmid (Abbink et al., J. Virol. 81(9):4654-63 (2007)) with flanking PacI sites was digested with PacI (fragment size 2108 bp). The two PacI and SbfI digested PCR fragments from the previous steps were cloned into the pBR backbone, resulting in pBrSrfI-SbfI/MluI-rITR plasmid. This plasmid was then digested with SbfI and MluI. The Ad20-42-42 wild type genomic DNA was digested with SbfI and MluI and the fragment between genome nucleotide position 17742 and 33714 was isolated and cloned into the plasmid above. This resulted in pBrAd20-42-42 SrfI-rITR which carries the wild type Ad20-42-42 genome from position 15373 up to the last nucleotide of the rITR (35187).

Deletion of E3 region: With the aim of deleting the E3 region two PCRs were performed using pBrAd20-42-42 SrfI-rITR plasmid as a template. The first was designed upstream from the region to be deleted; the forward primer was covering a natural AscI site, while in the reverse primer an SpeI site was incorporated. The second PCR was designed downstream to the E3 region. The reverse primer covered a natural EcoRI site in the Ad20-42-42 genome, while in the forward primer an SpeI site was incorporated. Afterwards the first product was double digested with SpeI and AscI and the second product with SpeI and EcoRI. The pBrAd20-42-42 Srf-rITR plasmid was digested with AscI and EcoRI and a 3-point ligation was performed with the digested plasmid and PCR products. The fragment between 26673 and 30753 genomic nucleotide position of the wild type Ad20-42-42 therefore fell out of the genome and the sequence was linked by the introduced SpeI site. This resulted in the pBrAd20-42-42.SrfI-rITR.dE3 plasmid.

Replacement of the native ORF6/7 by the homologue region of HAdV-5: To replace ORF6/7 three fragments were amplified by PCR. Two were designed to cover the region upstream and downstream from the ORF6/7 to be replaced. The forward primer of the first PCR carried an AscI site and the reverse primer of the second PCR carried an EcoRI site. A third PCR was performed to obtain the HAdV-5 ORF6/7 from a previously produced plasmid (pBr.Ad26.SrfI-rITR.dE3.5orf6 (Abbink et al., J. Virol. 81(9):4654-63 (2007)) and it was partly overlapping with the other two PCR products. These three PCR products were then subjected to fusion PCR and subsequently a double digestion with AscI and EcoRI. The pBrAd20-42-42.SrfI-rITR.dE3 plasmid was also digested with AscI and EcoRI and the digested fusion PCR product was inserted by a 2-point ligation to obtain the final right-end plasmid: pBrAd20-42-42 Srf-rITR.dE3.5orf6

### Generation and Production of Ad20-42-42-based Adenoviral Vectors

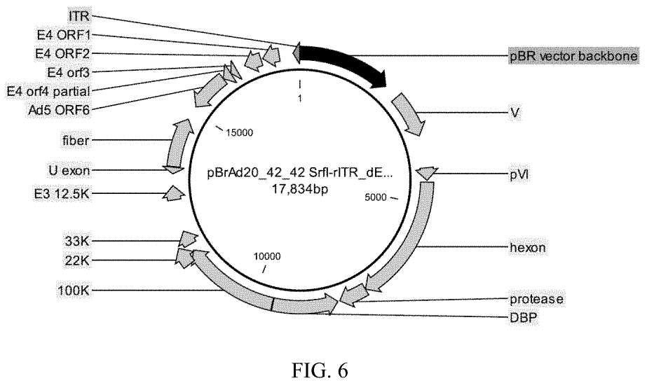

Adenoviral vectors Ad20-42-42.LacZ.5ORF6, Ad20-42-42.Luc+.5ORF6 and Ad20-42-42.eGFP.5ORF6, which respectively comprise adenoviral vector genome sequences SEQ ID NO:8, SEQ ID NO:9 and SEQ ID NO:10, were generated by transfection of the corresponding plasmids: (1) Adaptor plasmid “pAdApt20-42-42.LacZ (SEQ ID NO:3) or pAdApt20-42-42.Luc+ (SEQ ID NO:4) or pAdApt20-42-42.eGFP (SEQ ID NO:5); (2) intermediate plasmid “pBR.Ad20-42-42.SbfI final interm” (SEQ ID NO:6; FIG. 5); (3) right-end plasmid “pBrAd20-42-42 SrfI-rITR.dE3.5orf6” (SEQ ID NO:7; FIG. 6).

The transfection with the three-plasmid-system was performed in E1-complementing HEK293 cells. Prior to transfection into HEK293 cells, which were grown as adherent cultures in Dulbecco's modified Eagle's medium (DMEM) supplemented with 10% fetal bovine serum (FBS), the Ad vector genome plasmids were digested with Pad to release the respective adenoviral vector genome fragments from the plasmid. The transfections were performed according to standard procedures using Lipofectamine transfection reagent (Invitrogen; Carlsbad, Calif.). After harvesting of the viral rescue transfections, the viruses were further amplified by several successive infection rounds on HEK293 cell cultures. The viruses were purified from crude viral harvests using a two-step cesium chloride (CsCl) density gradient ultracentrifugation procedure as described before (Havenga et al., “Novel replication-incompetent adenoviral B-group vectors: high vector stability and yield in PER.C6 cells,” J. Gen. Virol. 87(8): 2135-43 (2006)). Viral particle (VP) titers were measured by a spectrophotometry-based procedure described previously (Maizel et al., “The polypeptides of adenovirus: I. Evidence for multiple protein components in the virion and a comparison of types 2, 7A, and 12,” Virology, 36(1):115-25 (1968)).

### Example 2

### Cellular and Humoral Immune Responses Induced by Novel Adenoviral Vector

This example describes experiments performed to assess the immunogenicity of the novel Ad20-42-42-based adenoviral vector generated herein. In these experiments, the novel vector was assessed for its ability to induce humoral and cellular immune responses against a vector-encoded (model) antigen in mice after intramuscular immunization. The vector tested expressed Firefly luciferase (FLuc) as a model antigen. The vector was compared side-by-side with a benchmark vector based on human adenovirus type 26 (HAdV-26, also referred to herein as Ad26). Immune responses against the respective antigens were measured using well-known immunological assays, such as an enzyme-linked immuno-pot assay (ELISPOT) and an enzyme-linked immunosorbent assay (ELISA).

### Cellular Immune Responses Induced by Ad20-42-42.FLuc

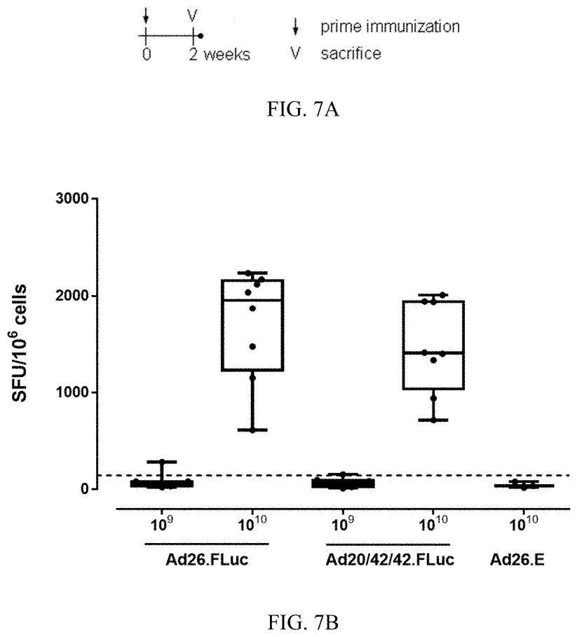

The immunogenicity of the novel adenoviral vector Ad20-42-42 was evaluated in Balb/C mice that were immunized by intramuscular injection with Ad26.FLuc (benchmark control), a Ad20-42-42 vector expressing firefly luciferase (Ad20-42-42.FLuc), or with an adenovector lacking a transgene (Ad26.E). Two vector doses were tested for administration: 10⁹ and 10¹⁰ viral particles (vp) per mouse, except for the Ad26.E group where 10¹⁰ vp was used. Animals were sacrificed two weeks post immunization and were sampled for serum and splenocytes (FIG. 7A).

Cellular immune responses against the vector-encoded antigen was evaluated by FLuc specific-IFN-γ ELISPOT assay. To this end, splenocytes that were sampled from immunized mice, at the time of sacrifice, were stimulated overnight with a 15mer overlapping FLuc peptide pool. The antigen specific immune responses were determined by measuring the relative number of IFN-γ-secreting cells (FIG. 7B). The results show that the cellular immune responses induced by Ad20-42-42 were comparable to the response seen for Ad26.Fluc at the highest immunization dose (10¹⁰). As expected, no FLuc specific responses were detected against the adenovectors lacking Firefly luciferase (Ad26.E). Overall, the data show that Ad20-42-42 induces a strong cellular response.

### Example 3

### Evaluation of Serological Cross-Neutralization Among Novel and Existing Adenoviral Vectors

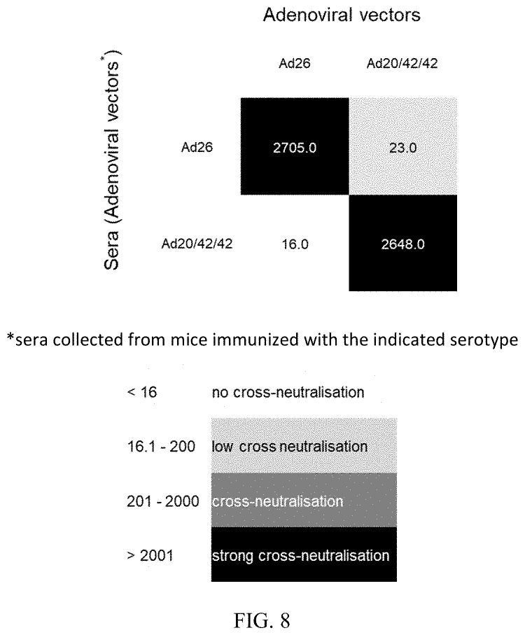

For their potential utility as new adenoviral vaccine vectors, the novel Ad20-42-42 adenoviral vector created herein would preferably be serologically distinct from existing adenoviral vectors currently in development as vaccine vectors, such as vectors based on human adenovirus serotype HAdV-26. Therefore, cross-neutralization tests were performed between the novel Ad20-42-42 adenoviral vector and an existing vector based on HAdV-26 (Ad26). To this end, mice antisera, raised against these vectors were cross-tested against both vectors in an adenovirus neutralization assay. The mice antisera used for this assay were collected from Balb/C mice, two weeks after their immunization with 10¹⁰ vector particles per mouse. The adenovirus neutralization assay was carried out as described previously (Spangers et al 2003. J. Clin. Microbiol. 41:5046-5052). Briefly, starting from a 1:16 dilution, the sera were 2-fold serially diluted, then pre-mixed with the adenoviral vectors expressing firefly luciferase (FLuc), and subsequently incubated overnight with A549 cells (at a multiplicity of infection (MOI) of 500 virus particles per cell). Luciferase activity levels in infected cell lysates measured 24 hours post-infection represented vector infection efficiencies. Neutralization titers against a given vector were defined as the highest serum dilution capable of giving a 90% reduction of vector infection efficiency. The neutralization titers were arbitrarily divided into the following categories: <16 (no neutralization), 16.1 to 200 (slightly cross-neutralizing), 201 to 2,000 (cross-neutralizing), and >2,001 (strongly cross-neutralizing). The results show no major cross-neutralization between the vectors tested (FIG. 8). There was only some slight, one-way cross-neutralization seen between vectors Ad20-42-42 and Ad26, with Ad26 antiserum displaying a neutralization titer against Ad20-42-42 of 23 and Ad20-42-42 antiserum not showing any neutralization against Ad26. Thus, the new adenoviral vector Ad20-42-42 displayed low to non-existent cross-neutralization with the human adenoviral vector Ad26.

### Example 4

### Seroprevalence of Novel Adenoviral Vector in Human Populations

High levels of pre-existing anti-vector humoral immunity in vaccine target populations can hamper potential use of a novel adenoviral vector as an efficacious vaccine platform. Therefore, the Ad20-42-42 vector was evaluated for its seroprevalence in 103 human serum samples. The vector was tested for neutralization by the human serum samples by performing the CPE-based assay (for a wild type (wt) virus) and the reporter assay (for a Luc+ expressing virus)

Standard adenovirus neutralization assays were carried out as described in Example 3 and as described previously (Spangers et al 2003. J. Clin. Microbiol. 41:5046-5052). For the wild type adenovirus neutralization assay, sera were heat-inactivated at 55° C. for 15 minutes and diluted (½, ¼, ⅛, 1/16, or 1/32). Next, 50 μl of adenovirus stock, diluted to 200 cell culture-inhibiting does 50% (CCID₅₀), was added to the wells containing serum. On day 5 to 6, plates were analyzed by the MTT assay (Promega) for inhibition of CPE. Sera were scored positive for neutralization when the protection of CPE was >90%. The percentage of replication inhibition was calculated relative to positive and negative controls.

As indicated above, briefly, starting from a 1:16 dilution, the sera were 2-fold serially diluted, then pre-mixed with the adenoviral vectors expressing firefly luciferase (FLuc), and subsequently incubated overnight with A549 cells (at a multiplicity of infection of 500 virus particles per cell). Luciferase activity levels in infected cell lysates, measured 24 hours post-infection, represented vector infection efficiencies. Neutralization titers against a given vector were defined as the highest serum dilution capable of giving a 90% reduction of vector infection efficiency. The neutralization titers were arbitrarily divided into the following categories: <16 (no neutralization), 16 to 50, 50 to 200, 200 to 500, 500 to 1000, and >1000.

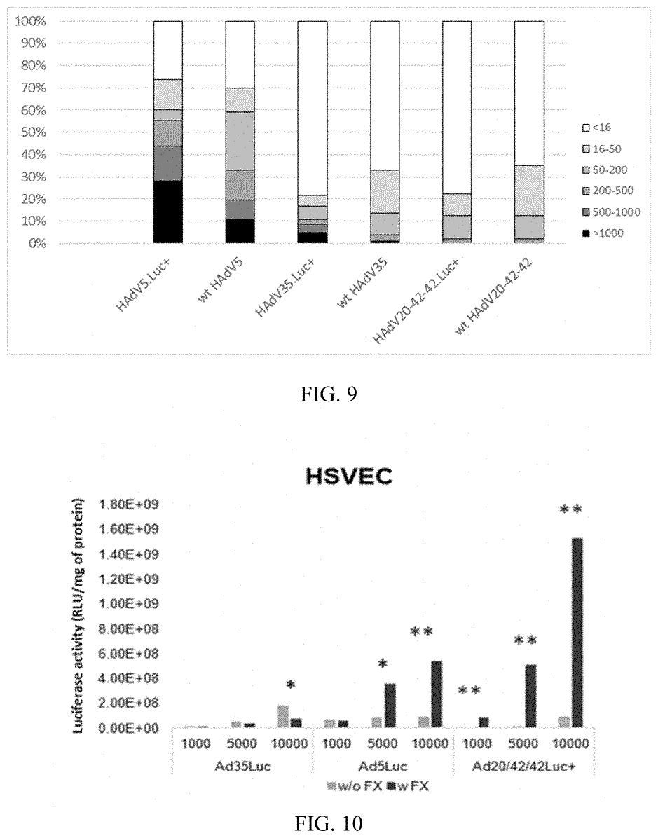

The results indicate that the Ad20-42-42 adenovirus vector has considerably low seroprevalence (20-35% seropositivity) in the studied human subjects, when compared to the HAdV-5 vector tested in the same assays (FIG. 9). Moreover, the positive neutralization titers that were seen against the novel Ad20-42-42 vector were generally low, mostly not higher than 200. In contrast, the positive neutralization titers found against HAdV-5 were in the higher range, reaching above 1000. While vector HAdV-35 showed similar % of seropositivity to Ad20-42-42, the observed titers for HAdV-35 were in higher ranges than those for Ad20-42-42.

Altogether, the above data indicate that pre-existing humoral anti-vector immunity against Ad20-42-42 vector can be considered low in the evaluated vaccine target populations, suggesting that this vector has potential as an efficacious vaccine vector in this human population.

### Example 5

### Transduction Capacity of Ad20-42-42 Vector in Human Vascular Cells

The ability to transduce cells of interest and to express the proteins they encode are essential features of vectors that are to be used in gene therapy. The novel adenoviral vector Ad20-42-42 was tested for transduction capacity in vascular cells using a luciferase assay.

Briefly, HSVEC (human saphenous vein endothelial cells) cells were transduced with a luciferase and LacZ-expressing Ad20-42-42 vector in a dose-dependent manner and in the presence or absence of blood coagulation factor X (FX) to ascertain sensitivity to FX-mediated modification of tropism or no. HAd5 and HAd35 were used as control vectors. Cells were plated at density of 10,000 cells/well in 96-well plates. They were infected with 1000, 5000 and 10,000 viral particles (vp) per cell of Ad5Luc, Ad35Luc and Ad20-42-42Luc+, expressing luciferase for 3 hours at 37° C. with and without adding FX. After the 3-hour incubation, medium was removed, and cells were cultured for 48 hours in a complete medium before the analysis of luciferase transgene expression was performed and expressed as relative light units (RLU) per milligram (mg) of protein.

Statistical significance between groups was calculated using two-sample, two-tailed Student's t-tests, where p<0.05 was considered statistically significant (p<0.05*, p<0.01**). All results represent averaged data from several experiments, with four replicates for each condition (FIG. 10).

The data obtained show a notably higher transduction potential of Ad20-42-42 over HAd5 and HAd35, in the presence of FX. The highest luciferase expression was noticed when a 10,000 vp/cell dose was applied, reaching levels of 1.5×10⁹ in the presence of FX, and ˜1×10⁸ when FX was absent. Thus, these results indicate that Ad20-42-42 transduction was significantly enhanced by FX and that this vector has stronger transduction properties over two control vectors, in the presence of FX.

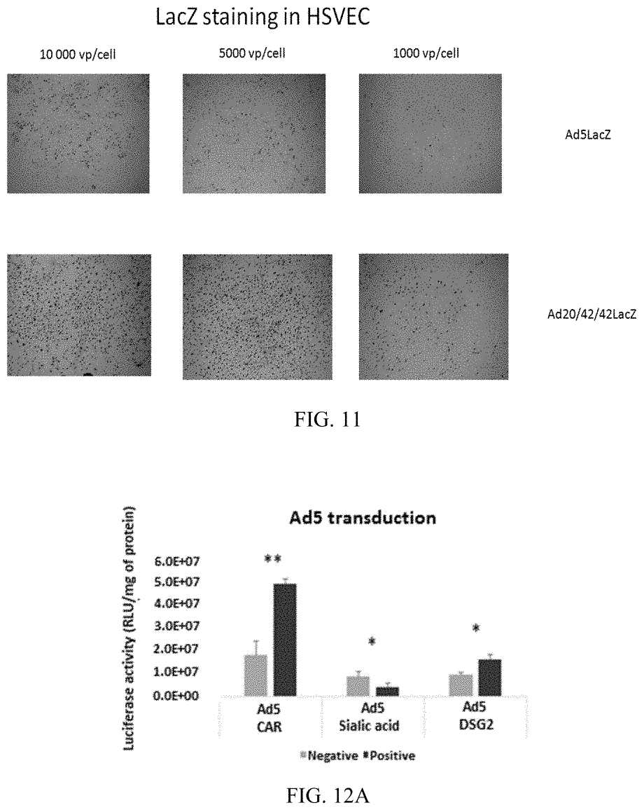

The superior transduction profile of Ad20-42-42 over HAd5 control vector was confirmed by LacZ staining of HSVEC cells, using three different vector doses (1000, 5000 and 10,000 vp/cell), in the presence of FX. The staining was observed with a simple light microscope (FIG. 11).

As expected, the results showed a gradual increase of the staining as the dose was increased. The staining for 1000, 5000 and 10,000 vp/cell is shown from right to left on FIG. 11. The cells transduced with Ad20-42-42 demonstrated a stronger staining as compared to the HAd5 transduced cells.

Overall, the data showed that Ad20-42-42 was capable of transducing vascular cells and that this is higher than control vectors in the presence of FX. This makes Ad20-42-42, a good gene therapy vector candidate to be used in the treatment of diseases where endothelial cells feature, such as cardio-vascular disease or cancer.

### Example 6

### Candidate Receptors for AdV20-42-42

An important feature of Ad20-42-42 which distinguishes it among many other vectors is its potential of binding to both, the Coxsackievirus and adenovirus receptor (CAR) and the CD46 cell receptor, and its sensitivity to FX enhancement in transduction, thus, broadening the scope of cells and tissues in humans that would be available for gene therapy utilizing the Ad20-42-42 vector.

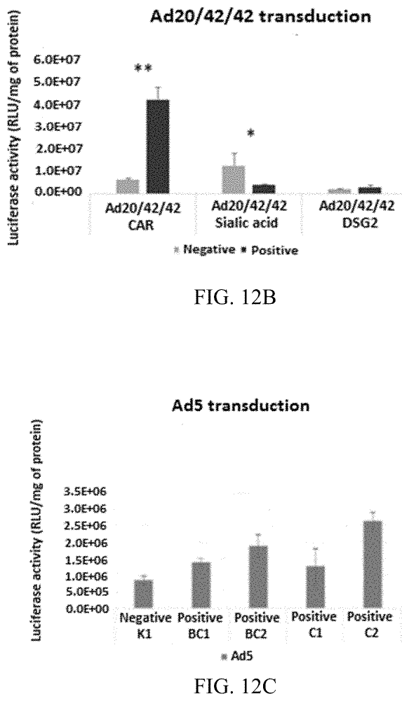

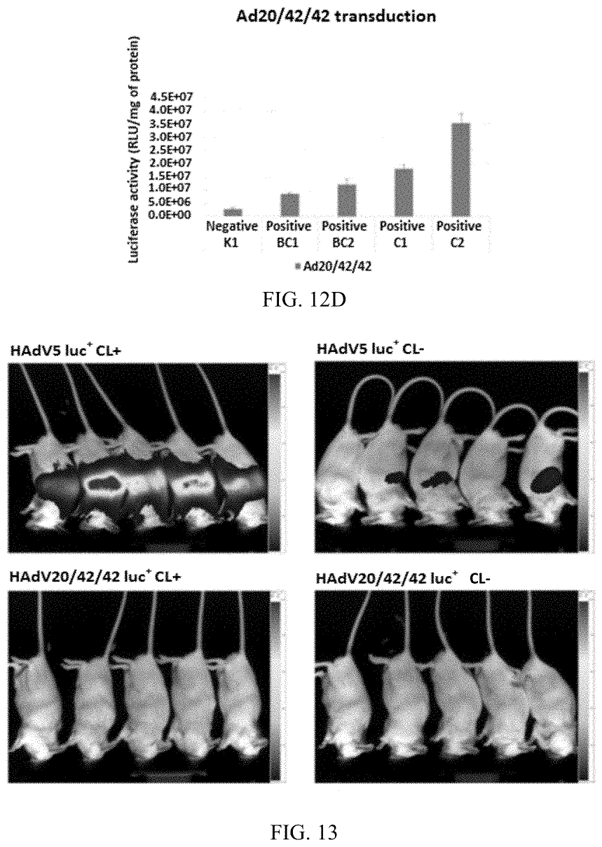

To examine the ability of Ad20-42-42 to bind to multiple receptors and use them as a tool for cell entry, several indicator cell types were used. Chinese hamster ovary (CHO) cells expressing or lacking CAR; CHO cells expressing or lacking sialic acid; and TC1 cells expressing or lacking desmoglein 2 (DSG2) were transduced with Ad20-42-42 and HAd5, which was used as a control vector (FIGS. 12A and 12B). The cells were transduced with 10,000 vp/cell, and a luciferase assay was performed as described above.

The results obtained from these experiments defined CAR as a potentially dominant transmembrane receptor to which Ad20-42-42 binds, as compared to sialic acid and DSG2, in cells having all three receptors on their surface.

Similar experiments were performed to examine the capability of this vector to bind to CD46, another receptor commonly used by many adenovirus types for cell entry. For this purpose, CHO cells containing or lacking different isoforms (BC1, BC2, C1 and C2) of this receptor were transduced with Ad20-42-42 and HAd5, which was used as a control vector. It was shown that Ad20-42-42 seems to predominantly use the C2 receptor isoform in the process of cell infection. On the other hand, K1 cells, lacking CD46, were poorly transduced (FIGS. 12C and 12D).

Statistical significance between groups was calculated using two-sample, two-tailed Student's t-tests, where p<0.05 was considered statistically significant (p<0.05*, p<0.01**). All results represent averaged data from several times performed experiments, with four replicates for each condition. Error bars are presented as standard error of the mean (SEM).

These findings indicate that the novel adenoviral vector Ad20-42-42 binds to both receptors, CAR and CD46, which are present in many cell types. This widens the range of cell types that could be transduced by the Ad20-42-42 vector, which may be beneficial to its use in gene therapy.

### Example 7

### Biodistribution of Ad20-42-42 in Mice

For the purpose of examining the biodistribution of Ad20-42-42, its effect on cardiovascular system (CVS) and its role as a gene delivery vector, in vivo experiments were performed.

Immunocompetent male mice, aged 8-10 weeks, were used. Six groups of animals were formed (each virus-testing group containing 5 animals and control PBS groups 3 animals). To deplete circulating macrophages and more efficiently evaluate the transit of the virus at the whole organism level, 200 μl of clodronate liposomes (CL) were intravenously (i.v.) administered to corresponding groups 48 hours prior to virus administration.

Treatment groups were i.v. infected with a single virus dose (10×10¹¹ virus particles (VP) diluted in 100 μl of PBS), of Ad20-42-42Luc+ or HAd5Luc, which was used as a control vector. Control groups instead were injected with 100 ml of PBS at the same time point.

48 hours post virus delivery, luciferase activity readout was performed using bioluminescent imaging, after 0.5 ml of luciferin had been injected into the animals. Animals were maintained under inhalational anesthesia. The levels of detected luciferase expression are shown in FIG. 13.

After the imaging was completed, animals were sacrificed and their organs (liver, heart, spleen, kidney, intestine, pancreas and lungs) were collected. Vector genomes were quantified by qPCR.

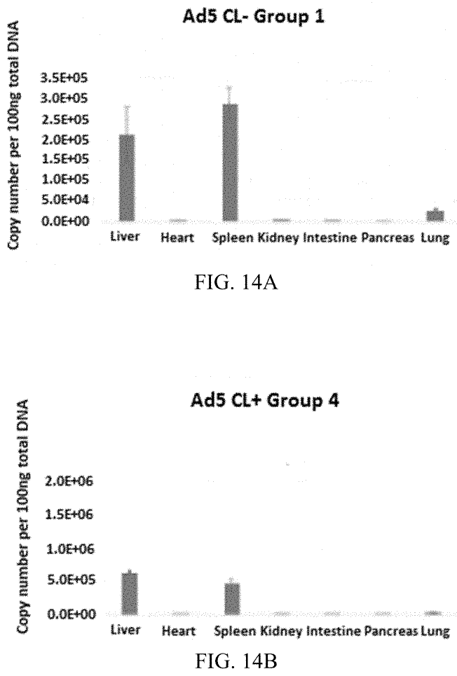

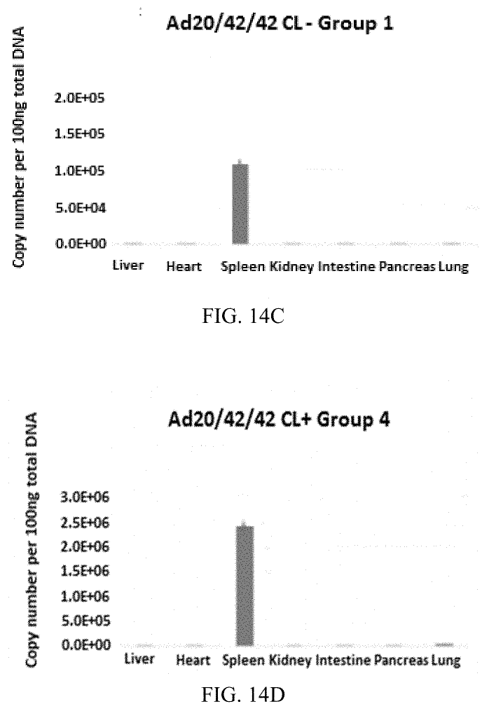

In the control group of animals infected with HAd5, virus was mainly distributed in liver and spleen at the levels of 2×10⁵ and ˜3×10⁵ bioluminescent units, respectively in a group not treated with CL, while in the group pre-treated with CL, distribution was even higher, closer to 5×10⁵ in both organs (FIGS. 14A-14B).

On the other hand, Ad20-42-42 appears to have only a spleen tropism, while in other organs it was not detected. As expected, the total DNA copy number was significantly higher when CL was added (˜2.5×10⁶) in comparison to the group without CL pretreatment (1×10⁵) (FIGS. 14C-14D).

All together, these data indicate that Ad20-42-42 has a good safety profile with only spleen tropism found in the studies, while a number of DNA copies found in other organs tested was poorly detectable. The Ad20-42-42 vector did not show any tropism to the liver, which makes it a vector with low liver availability and toxicity, and, therefore, more suited for gene therapy where gene transfer to the liver would be a hindrance to efficacy in the target tissue.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated nucleic acid sequence encoding a chimeric adenoviral capsid or a functional derivative thereof, wherein the chimeric adenoviral capsid or functional derivative thereof comprises a fiber polypeptide having an amino acid sequence with at least 95% identity to the amino acid sequence of SEQ ID NO:11, a hexon polypeptide having an amino acid sequence with at least 95% identity to the amino acid sequence of SEQ ID NO:12, and a penton polypeptide having an amino acid sequence with at least 95% identity to the amino acid sequence of SEQ ID NO:13.

2. The isolated nucleic acid sequence of claim 1, wherein the fiber polypeptide sequence comprises the amino acid sequence of SEQ ID NO:11.

3. The isolated nucleic acid of claim 1, wherein the hexon polypeptide sequence comprises the amino acid sequence of SEQ ID NO:12.

4. The isolated nucleic acid of claim 1, wherein the penton polypeptide comprises the amino acid sequence of SEQ ID NO:13.

5. A vector comprising the isolated nucleic acid of claim 1.

6. The vector of claim 5, wherein the vector is an adenoviral vector.

7. The vector of claim 6, wherein the adenoviral vector further comprises a transgene.

8. The vector of claim 6, wherein the adenoviral vector further comprises an E1 deletion and/or an E3 deletion.

9. (canceled)

10. The vector of claim 6, wherein the adenoviral vector is a chimeric adenoviral vector comprising one or more adenoviral nucleic acid sequences from at least one of human adenovirus-4, human adenovirus-5, human adenovirus-26, or human adenovirus-35.

11. The vector of claim 10, wherein the adenoviral vector comprises a human adenovirus-5 (HAdV-5) E4 orf6.

12. The vector of claim 6, wherein the adenoviral vector comprises a nucleic acid sequence selected from the group of SEQ ID NO:8, SEQ ID NO:9, and SEQ ID NO:10.

13. The vector of claim 7, wherein the transgene is located at the E1 deletion, at the E3 deletion, and/or adjacent to the right inverted terminal repeat (rITR).

14. A recombinant cell comprising the vector of claim 5.

15. A method of producing a vector, comprising:
(a) growing the recombinant cell of claim 14 under conditions for production of the adenoviral vector; and
(b) isolating the vector from the recombinant cell.

16. An immunogenic composition comprising the adenoviral vector of claim 6 and a pharmaceutically acceptable carrier.

17. A method of inducing an immune response in a subject in need thereof, the method comprising administering to the subject the immunogenic composition of claim 16.

18. A method of producing a vaccine, the method comprising combining an adenoviral vector of claim 6 with a pharmaceutically acceptable carrier.

19. A method of expressing a transgene in a subject in need thereof, the method comprising:
a. identifying a subject in need of the expressed transgene;
b. contacting the subject with the vector of claim 7; and
c. expressing the transgene in the subject.

20. The method of claim 19, wherein expression of the transgene in the subject in need thereof treats or prevents a disease or disorder.

21. The method of claim 19, wherein contacting the subject with the vector comprises isolating a cell from the subject and contacting the cell with the vector.

22. The method of claim 19, wherein the subject in need thereof is a human subject.
